(19) United States
(12) Patent Application Publication
(10) Pub. No.: US 2006/0200011 A1
(43) Pub. Date: Sep. 7, 2006

(54) Health management apparatus, health management system, health management method and computer program product

(75) Inventors: Takuji Suzuki, Kanagawa (JP); Kenichi Kameyama, Kanagawa (JP); Akihisa Moriya, Kanagawa (JP); Kazushige Ouchi, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kabushiki Kaisha Toshiba, Minato-ku (JP)

(21) Appl. No.: 11/345,292

(22) Filed: Feb. 2, 2006

(30) Foreign Application Priority Data
Feb. 3, 2005 (JP) 2005-028184

(51) Int. Cl. A61B 5/00; A61B 5/02

(52) U.S. Cl. 600/301; 600/500

(57) ABSTRACT

A health management apparatus includes a first pulse wave measuring unit that measures a first pulse wave of a subject during sleep; and a second pulse wave measuring unit that measures a second pulse wave of the subject during sleep. The second pulse wave is different from the first pulse wave in propagation time from the heart of the subject. The apparatus also includes a pulse transmission time calculating unit that calculates a pulse transmission time indicating a time difference between the first and second pulse waves; a pulse interval calculating unit that calculates a pulse interval based on at least one of the first and second pulse waves; an autonomic nerve index calculating unit that calculates an autonomic nerve index based on the pulse transmission time and the pulse interval; and a health determining unit that determines the condition of health of the subject based on the autonomic nerve index.

### CROSS-REFERENCE TO RELATED APPLICATIONS

This application is based upon and claims the benefit of priority from the prior Japanese Patent Application No. 2005-028184, filed on Feb. 3, 2005; the entire contents of which are incorporated herein by reference.

### BACKGROUND OF THE INVENTION

1. Field of the Invention

The present invention relates to an apparatus, a system, a method, and a computer program product for managing the condition of health of a subject being tested by measuring the pulse wave of the subject during sleep.

2. Description of the Related Art

There have been sleep determining devices that determine the sleep states of subjects based on the pulse interval data representing each cycle of the pulse wave of each subject and the movement data indicating the body movement of each subject. Such a sleep determining device that can readily assess a sleep state in everyday life is more advantageous than a large-scale device that automatically assesses a sleep state from a body signal pattern called polysomnogram showing brain waves, eye movement, myoelectric activities, and electrocardiogram activities, and therefore it is researched and developed.

In this sleep determining device, the intervals of heartbeat that is an autonomic nerve activity during sleep are set as the intervals of pulse waves, and a sleep state is assessed based on an autonomic nerve index obtained from a pulse interval variation. For example, pulse waves that show changes in blood flow in the blood vessels of the hands vary with the heartbeat. Accordingly, the heartbeat intervals can be determined from the pulse intervals.

Japanese Patent Application Laid-Open No. 2002-291710 discloses a technique of assessing a sleep state based on an autonomic nerve index that is obtained from the frequency spectrum components of pulse wave data, for example. More specifically, a series of pulse interval data is obtained from pulse wave data, and is then converted into a frequency spectrum distribution. An autonomic nerve index is obtained from the power spectrum values in the low-frequency region (LF: in the neighborhood of 0.05 Hz to 0.15 Hz) and the high-frequency region (HF: in the neighborhood of 0.15 Hz to 0.4 Hz) determined from the series of pulse interval data converted into the frequency spectrum distribution. Based on the autonomic nerve index, a sleep state is assessed.

Also, there has been the problem of blood pressure variabilitys during sleep. If early-morning high blood pressure is detected, for example, the probability of cerebral stroke before wake-up is more than three times as high. Also, during REM sleep, a so-called “autonomic storm” phenomenon is caused, and unexpected high blood pressure is observed due to great changes in the autonomic nerves. In the case of a sleep apnea syndrome, a hypoxic state leads to a blood pressure rise, and when the subject resumes breathing, the blood pressure rapidly rises due to sympathetic hypertonia from overbreathing.

However, when blood pressure is measured at a hospital or the like, high blood pressure might be observed due to tension during the examination, which is called “white coat hypertension.” This makes it difficult to obtain an accurate blood pressure value.

As the means of continuously measuring blood pressure, a portable automatic blood pressure monitor (manufactured as a medical instrument by A&D Co., Ltd.) is available on the market. This is a portable version of a conventional blood pressure monitor with cuffs. This blood pressure monitor measures blood pressure by automatically operating the cuffs at predetermines times that are set by the clock contained in the device. However, the constriction by the cuffs is very uncomfortable, and becomes a hindrance to everyday life, especially to sleep.

To counter this problem, blood pressure monitors for measuring blood pressure without cuffs have become available on the market. For example, a blood pressure monitor manufactured by Casio Computer Co., Ltd. measures blood pressure based on the inverse relationship between the pulse transmission time and the blood pressure. Electrodes are attached to the surface and the back of the wristwatch, and a LED and a photodiode for measuring pulse waves are placed in the center of the electrode on the surface. By putting a finger on the electrode, an electrocardiogram and a pulse wave can be simultaneously measured, and the blood pressure can be determined from the pulse transmission time. However, since a finger must be put on the surface of the wristwatch, it is not suited for continuous blood pressure measurement.

Also, a conventional sleep determining device can assess a sleep state such as arousal, REM sleep, non-REM sleep, or arousal during sleep. However, pulse wave data is obtained by measuring the pulse waves that represent changes in blood flow in the blood vessels of the hand. As a result, the pulse wave data tends to be affected by movement of the body parts such as the hands and the legs, and the accuracy in sleep assessment becomes poor.

### SUMMARY OF THE INVENTION

According to one aspect of the present invention, a health management apparatus includes a first pulse wave measuring unit that measures a first pulse wave of a subject during sleep; and a second pulse wave measuring unit that measures a second pulse wave of the subject during sleep. The second pulse wave is different from the first pulse wave in propagation time from the heart of the subject. The apparatus also includes a pulse transmission time calculating unit that calculates a pulse transmission time indicating a time difference between the first pulse wave and the second pulse wave; a pulse interval calculating unit that calculates a pulse interval based on at least one of the first pulse wave and the second pulse wave; an autonomic nerve index calculating unit that calculates an autonomic nerve index indicating an autonomic nerve activity of the subject based on the pulse transmission time and the pulse interval; and a health determining unit that determines the condition of health of the subject based on the autonomic nerve index.

According to another aspect of the present invention, a health management apparatus includes a first pulse wave measuring unit that measures a first pulse wave of a subject during sleep; and a second pulse wave measuring unit that measures a second pulse wave of the subject during sleep. The second pulse wave is different from the first pulse wave in propagation time from the heart of the subject. The apparatus also includes a pulse transmission time calculating unit that calculates a pulse transmission time indicating a time difference between the first pulse wave and the second pulse wave; a pulse interval calculating unit that calculates a pulse interval based on at least one of the first pulse wave and the second pulse wave; an autonomic nerve index calculating unit that calculates an autonomic nerve index indicating an autonomic nerve activity of the subject based on the pulse transmission time and the pulse interval; a blood pressure value calculating unit that calculates a blood pressure value based on the pulse transmission time; and a health determining unit that determines the condition of health of the subject based on the autonomic nerve index and the blood pressure value.

According to still another aspect of the present invention, a health management apparatus includes an electrocardiogram activity measuring unit that measures the degree of an electrocardiogram activity of a subject during sleep; a first pulse wave measuring unit that measures a first pulse wave of the subject during sleep; a pulse transmission time calculating unit that calculates a pulse transmission time indicating a time difference between the electrocardiogram activity and the first pulse wave; a pulse interval calculating unit that calculates a pulse interval based on at least one of the degree of the electrocardiogram activity and the first pulse wave; an autonomic nerve index calculating unit that calculates an autonomic nerve index indicating an autonomic nerve activity of the subject based on the pulse transmission time and the pulse interval; and a health determining unit that determines the condition of health of the subject based on the autonomic nerve index.

According to still another aspect of the present invention, a health management apparatus includes an electrocardiogram activity measuring unit that measures the degree of an electrocardiogram activity of a subject during sleep; a first pulse wave measuring unit that measures a first pulse wave of the subject during sleep; a pulse transmission time calculating unit that calculates a pulse transmission time indicating a time difference between the electrocardiogram activity and the first pulse wave; a pulse interval calculating unit that calculates a pulse interval based on at least one of the degree of the electrocardiogram activity and the first pulse wave; an autonomic nerve index calculating unit that calculates an autonomic nerve index indicating an autonomic nerve activity of the subject based on the pulse transmission time and the pulse interval; a blood pressure value calculating unit that calculates a blood pressure value based on the pulse transmission time; and a health determining unit that determines the condition of health of the subject based on the autonomic nerve index and the blood pressure value.

According to still another aspect of the present invention, a health management system includes a health management main apparatus and a health management sub apparatus that manage the condition of health of a subject. The health management sub apparatus includes a first pulse wave measuring unit that measures a first pulse wave of the subject during sleep; and a transmission unit that transmits the first pulse wave to the health management main apparatus. The health management main apparatus includes a reception unit that receives the first pulse wave from the health management sub apparatus; a second pulse wave measuring unit that measures a second pulse wave of the subject during sleep, the second pulse wave being different from the first pulse wave in propagation time from the heart of the subject; a pulse transmission time calculating unit that calculates a pulse transmission time indicating a time difference between the first pulse wave and the second pulse wave; a pulse interval calculating unit that calculates a pulse interval based on at least one of the first pulse wave and the second pulse wave; an autonomic nerve index calculating unit that calculates an autonomic nerve index indicating an autonomic nerve activity of the subject based on the pulse transmission time and the pulse interval; and a health determining unit that determines the condition of health of the subject based on the autonomic nerve index.

According to still another aspect of the present invention, a health management system includes a health management main apparatus and a health management sub apparatus that manage the condition of health of a subject. The health management sub apparatus includes a first pulse wave measuring unit that measures a first pulse wave of the subject during sleep; and a transmission unit that transmits the first pulse wave to the health management main apparatus. The health management main apparatus includes a reception unit that receives the first pulse wave from the health management sub apparatus; a second pulse wave measuring unit that measures a second pulse wave of the subject during sleep, the second pulse wave being different from the first pulse wave in propagation time from the heart of the subject; a pulse transmission time calculating unit that calculates a pulse transmission time that is the time difference between the first pulse wave and the second pulse wave; a pulse interval calculating unit that calculates a pulse interval based on at least one of the first pulse wave and the second pulse wave; an autonomic nerve index calculating unit that calculates an autonomic nerve index indicating an autonomic nerve activity of the subject based on the pulse transmission time and the pulse interval; a blood pressure value calculating unit that calculates a blood pressure value, based on the pulse transmission time; and a health determining unit that determines the condition of health of the subject based on the autonomic nerve index and the blood pressure value.

According to still another aspect of the present invention, a health management method includes measuring a first pulse wave of a subject during sleep; measuring a second pulse wave of the subject during sleep, the second pulse wave being different from the first pulse wave in propagation time from the heart of the subject; calculating a pulse transmission time indicating a time difference between the first pulse wave and the second pulse wave; calculating a pulse interval, based on at least one of the first pulse wave and the second pulse wave; calculating an autonomic nerve index indicating an autonomic nerve activity of the subject based on the pulse transmission time and the pulse interval; and determining the condition of health of the subject, based on the autonomic nerve index.

According to still another aspect of the present invention, a health management method includes measuring a first pulse wave of a subject during sleep; measuring a second pulse wave of the subject during sleep, the second pulse wave being different from the first pulse wave in propagation time from the heart of the subject; calculating a pulse transmission time indicating a time difference between the first pulse wave and the second pulse wave; calculating a pulse interval based on at least one of the first pulse wave and the second pulse wave; calculating an autonomic nerve index indicating an autonomic nerve activity of the subject based on the pulse transmission time and the pulse interval; calculating a blood pressure value, based on the pulse transmission time; and determining the condition of health of the subject, based on the autonomic nerve index and the blood pressure value.

A computer program product according to still another aspect of the present invention causes a computer to perform any one of the health management methods according to the present invention.

### BRIEF DESCRIPTION OF THE DRAWINGS

FIG. 1 shows the entire structure of a sleep-time health management apparatus according to a first embodiment of the present invention;

FIG. 2 schematically shows the health management apparatus shown in FIG. 1;

FIG. 3 shows an example of how the health management apparatus is attached to a subject;

FIG. 4 shows the pulse transmission time;

FIG. 5 shows the operation of an autonomic nerve index calculating unit;

FIG. 6 shows the operation of calculating LF from blood pressure values;

FIG. 7 is a flowchart of the sleep-time health managing operation of the health management apparatus;

FIG. 8 is a flowchart of the operation performed in step S152 of the flowchart of FIG. 7;

FIG. 9 is a flowchart of the sleep-time health managing operation of a health management apparatus according to a second embodiment of the present invention;

FIG. 10 is a flowchart of the operation performed in step S152 of the flowchart according to the second embodiment;

FIG. 11 is a block diagram showing the entire structure of a health management apparatus according to a third embodiment of the present invention;

FIG. 12 schematically shows the data structure in a measured value storing unit;

FIG. 13 schematically shows the result of accumulation by a measured value accumulating unit;

FIG. 14 shows the blood pressure values in association with the sleep states and the autonomic nerve activities;

FIG. 15A schematically shows a blood variation pattern;

FIG. 15B schematically shows another blood variation pattern;

FIG. 15C schematically shows still another blood variation pattern;

FIG. 16 shows an example of the screen to be displayed on a display unit when the pattern of FIG. 15A is detected;

FIG. 17 shows the operation of calculating the rate of change of the pulse transmission time;

FIG. 18 is a graph showing the number of times counted by the hour (the blood pressure increase frequency);

FIG. 19 shows an example of the screen displaying the states of sleep as well as blood pressure variability patterns on the display unit;

FIG. 20 is an example of a table displayed on the display unit showing accumulated values stored in a reference blood pressure storing unit;

FIG. 21 shows an example of how a health management system according to a fourth embodiment of the present invention is attached to a subject being tested;

FIG. 22 is a block diagram showing the entire structure of the health management system according to the fourth embodiment;

FIG. 23 shows an example of how a health management system according to a fifth embodiment of the present invention is attached to a subject being tested; and

FIG. 24 is a block diagram showing the entire structure of the health management system according to the fifth embodiment.

### DETAILED DESCRIPTION OF THE INVENTION

Exemplary embodiments of a health management apparatus, a health management system, a health management method, and a computer program product of the present invention will be described in detail below with reference to the accompanying drawings.

A health management apparatus according to a first embodiment of the present invention measures a change in blood pressure, using an electrocardiogram sensor as well as a pulse wave sensor. The health management apparatus adds the obtained value of the blood-pressure change to parameters, and estimates a sleep state.

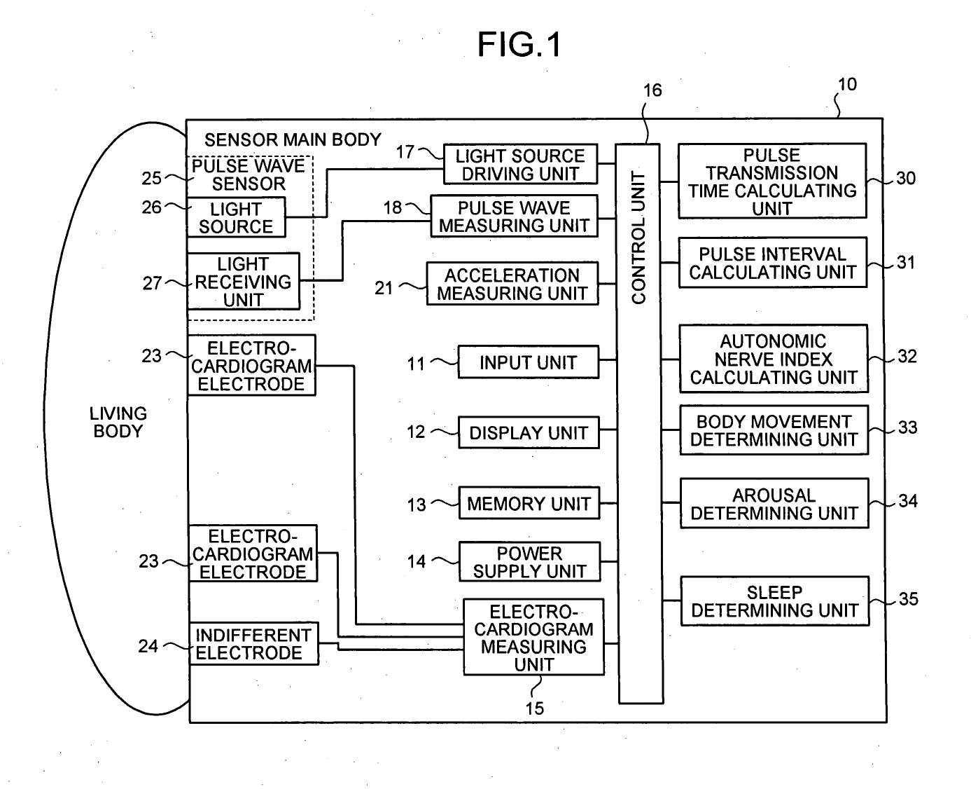

FIG. 1 shows the entire structure of the health management apparatus according to the first embodiment. As shown in FIG. 1, the health management apparatus 10 includes an input unit 11, a display unit 12, a memory unit 13, a power supply unit 14, an electrocardiogram measuring unit 15, a control unit 16, a light source driving unit 17, a pulse wave measuring unit 18, an acceleration measuring unit 21, electrocardiogram electrodes 23a and 23b, an indifferent electrode 24, a pulse wave sensor 25, a pulse transmission time calculating unit 30, a pulse interval calculating unit 31, an autonomic nerve index calculating unit 32, a body movement determining unit 33, an arousal determining unit 34, and a sleep determining unit 35.

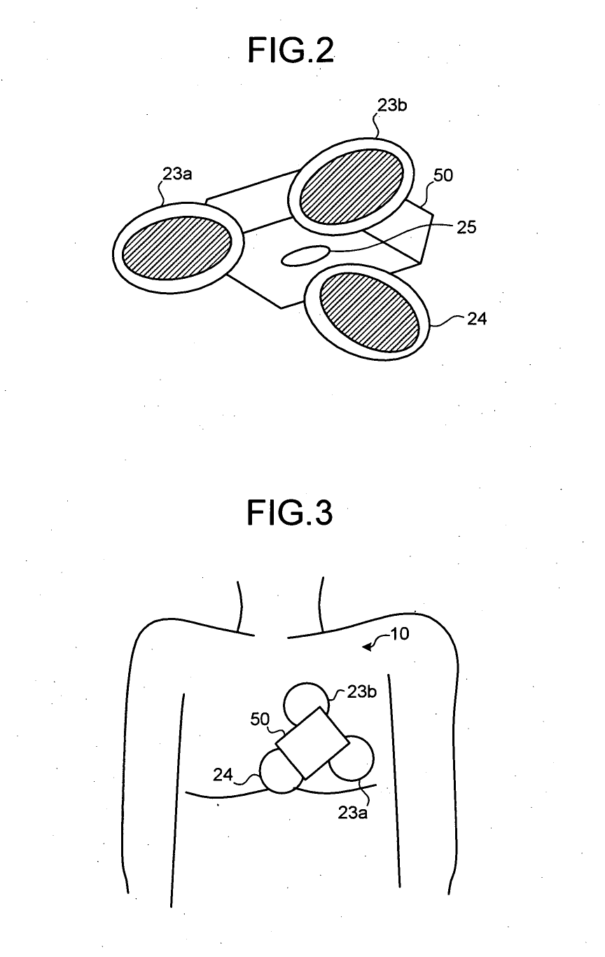

How the health management apparatus 10 of FIG. 1 is attached to a subject being tested is now described. FIG. 2 is an external view of the health management apparatus 10 of FIG. 1. FIG. 3 shows an example of how the health management apparatus 10 is attached to a subject being tested. As shown in FIG. 2, the pulse wave sensor 25 is located at the center of the housing 50 of the main body. The two electrocardiogram electrodes 23a and 23b and the indifferent electrode 24 are disposed at the ends of the housing 50.

As shown in FIG. 3, the health management apparatus 10 is attached to the chest region of the subject being tested, for example. Here, the electrocardiogram electrodes 23a and 23b are attached to two spots on the skin, with the heart being located between the two spots. The indifferent electrode 24 should preferably be attached to a spot as far as possible from the heart.

Referring back to FIG. 1, the input unit 11 is a switch for a user to turn ON and OFF the power supply and to issue a request or an instruction to switch displays. The display unit 12 is a display device to display the result of sleep determination, and, to be more specific, is formed with an LCD or the like.

The memory unit 13 stores measurement data such as pulse wave data, Electrocardiogram data, and body movement data, post-processing data such as pulse interval data, and threshold data such as threshold values for determining a sleep state. To be more specific, the memory unit 13 is a flash memory or the like. The power supply unit 14 supplies power to the health management apparatus 10, and, to be more specific, is a battery.

The control unit 16 controls the timing of electrocardiogram and pulse wave measurement, and accumulates and processes received data.

The acceleration measuring unit 21 measures acceleration data as the body movement data indicating the body movement of the subject being tested, and performs data conversion. The acceleration sensor is an accelerometer sensor that measures accelerations of −2 g to 2 g in the three axis directions, and is mounted onto the main body of the health management apparatus 10. After adjusting the gain and offset of analog data of the acceleration sensor with an adjusting circuit, the acceleration measuring unit 21 converts the analog data into digital data with a 10-bit A-D converter. The converted data is then output to the control unit 16.

The electrocardiogram measuring unit 15 measures the potential difference between the indifferent electrode 24 and the two electrocardiogram electrodes 23a and 23b for measuring the electrocardiogram. The electrocardiogram measuring unit 15 performs processing such as amplifying or filtering on the potential difference. The electrocardiogram measuring unit 15 then A-D converts the potential difference. The electrocardiogram measuring unit 15 is formed with an electronic circuit for transferring the converted data to the control unit 16.

The electrocardiogram measuring unit 15 measures the electrocardiogram in the same timing as the pulse wave measuring unit 18 measuring the pulse wave.

The pulse wave sensor 25 includes a light source 26 that is a blue LED and a light receiving unit 27 that is a photodiode. The pulse wave sensor 25 irradiates the skin surface with light, and, using the photodiode, captures the variation of reflection light that varies with changes in blood flow in blood capillaries.

The pulse wave measuring unit 18 measures the pulse wave data of the subject being tested, and performs data conversion on the data. Using a current-voltage converter, the pulse wave measuring unit 18 converts the output current from the photodiode of the pulse wave sensor 25 into a voltage. Using an amplifier, the pulse wave measuring unit 18 amplifies the voltage, which is then subjected to high-pass filtering (cutoff frequency: 0.1 Hz) and low-pass filtering (cutoff frequency: 50 Hz). The pulse wave measuring unit 18 then converts the voltage to a digital value with a 10-bit A-D converter. The converted data is output to the control unit 16.

The electrocardiogram measuring unit 15 and the pulse wave measuring unit 18 perform electrocardiogram measurement and pulse wave measurement in the same timing.

When a blue LED is used as the light source 26, the light source driving unit 17 serves as a voltage supply unit for driving the blue LED.

The pulse transmission time calculating unit 30 calculates the pulse transmission time, based on the potential difference measured by the electrocardiogram measuring unit 15, which is the peak of the electrocardiogram action potential, and the peak of the pulse wave measured by the pulse wave measuring unit 18.

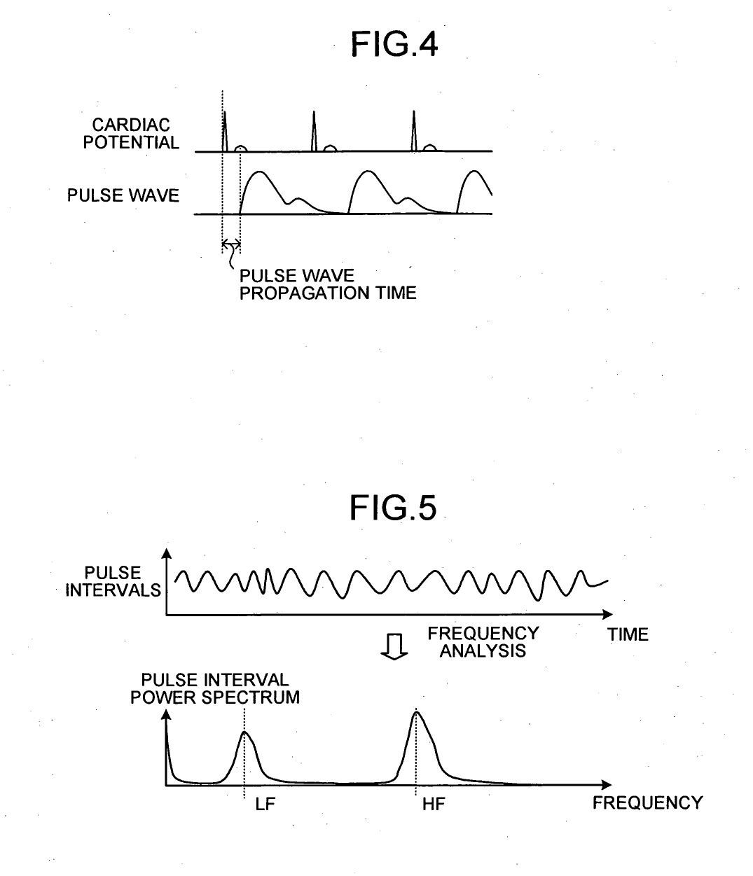

Here, the pulse transmission time is explained. FIG. 4 shows the pulse transmission time for better understanding. The electrocardiogram measuring unit 15 directly measures the electrocardiogram action potential. Accordingly, the real-time action of the heart can be measured. On the other hand, there is a time lag in the measurement of the pulse wave in the periphery, as the pulse wave flows to the peripheral blood vessels via arteries. The time lag is called the pulse transmission time. The pulse transmission time reflects the blood flow, and is inversely proportional to the blood pressure. Accordingly, changes in blood pressure can be detected from changes in the pulse transmission time.

The pulse interval calculating unit 31 calculates the pulse interval from the pulse wave measured by the pulse wave measuring unit 18. Here, the “pulse interval” is the period of one cycle of the pulse wave.

More specifically, a series of pulse wave data is sampled from the pulse wave measured by the pulse wave measuring unit 18. The sampled series of pulse wave data is time-differentiated to remove the DC variation of the series of pulse wave data.

The minimum and maximum values of the pulse wave data of approximately one second before and after the processing point in the series of pulse wave data without the DC variation component are obtained, and a predetermined value between the minimum and maximum values is set as the threshold value. The threshold value is preferably a value that is 90% of the amplitude from the minimum value, with the amplitude being the difference between the minimum and maximum values.

Based on the series of pulse wave data from which the DC variation component has been removed, the time at which the value corresponding to the threshold value appears in the series of pulse wave data is calculated, and the calculated time intervals are set as pulse intervals.

The pulse interval data is irregular interval data. To carry out a frequency analysis, the pulse interval data needs to be converted into equal interval data. Therefore, interpolation and re-sampling are performed on the irregular pulse interval data, so as to generate equal pulse interval data. For example, using three sampling points before and after the point to be interpolated by a cubic polynomial interpolation method, equal pulse interval data is generated.

The autonomic nerve index calculating unit 32 calculates two autonomic nerve indexes: an index LF of a low-frequency region (in the neighborhood of 0.05 Hz to 0.15 Hz) for determining a sleep state; and an index HF of a high-frequency region (in the neighborhood of 0.15 Hz to 0.4 Hz). FIG. 5 shows the operation of the autonomic nerve index calculating unit 32.

First, the equal pulse interval data is converted into a frequency spectrum distribution through FFT (fast Fourier transform). Next, the index LF and the index HF are obtained from the frequency spectrum frequency. More specifically, the index LF and the index HF are obtained from the arithmetic mean values of the three points: the peak of power spectra and two points located at equal distances in front of and behind the peak. The autonomic nerve index calculating unit 32 further calculates the index LF based on the pulse transmission time calculated by the pulse transmission time calculating unit 30.

When the index LF of a heart rate variation by this method, there is the problem of poor peak detection of the index LF due to body movement or fluctuation of the low-frequency region. To eliminate this problem, the autonomic nerve index calculating unit 32 uses the index LF calculated based on the pulse transmission time determined by the pulse transmission time calculating unit 30, so as to obtain the mean value of the three points in front of and behind the index LF.

In this embodiment, the FFT method is used as a frequency analysis method to reduce the trouble of data processing. However, it is also possible to use the AR model method, the maximum entropy method, the wavelet analysis method, or the like. Alternatively, an FFT method with smaller data processing load may be used.

The pulse transmission time is inversely proportional to the blood pressure, as expressed by equation (1):
Blood pressure=1/pulse transmission time×α (1)
where α is a constant.

At the start of measurement, the initial blood pressure is measured with a conventional blood-pressure meter. The constant α is calculated by assigning the measured value and the pulse transmission time to equation (1). From equation (1), the pulse transmission time is converted into the blood pressure value, and the index LF is determined from the blood pressure value.

By another method, a database for associating the normal blood pressure value of each subject to be tested with the pulse transmission time is employed. Based on the blood pressure value and the pulse transmission time stored in the database, the constant α can be calculated.

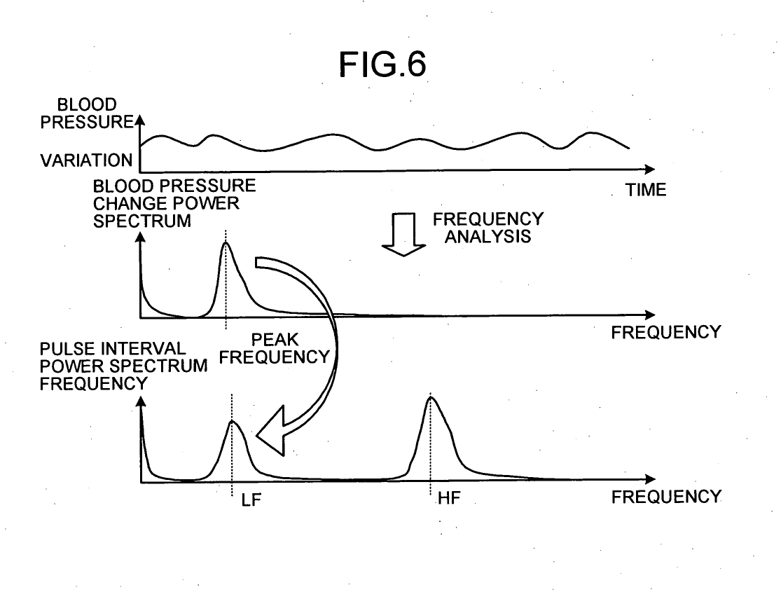

FIG. 6 shows the operation of calculating the index LF using the blood pressure value. As shown in FIG. 6, the index LF obtained through frequency analysis of blood pressure values is in synchronization with the index LF obtained from the pulse wave interval data.

Alternatively, the mean value of the peak frequency with respect to the heart rate variation and the peak frequency of the blood pressure variability may be used as the peak frequency of the index LF.

The body movement determining unit 33 time-differentiates the acceleration data of three axis directions obtained from the acceleration measuring unit 21, so as to obtain the differential coefficient of the accelerations of the three axis directions. The body movement determining unit 33 then determines the variation of body movement data that is the square root of the sum of squares of each differential coefficient of the accelerations of the three axis directions, and the amount of body movement that is the mean value of the variations of the body movement data in the pulse period. If the variation of the amount of body movement is greater than a predetermined threshold value, body movement is detected. For example, the predetermined threshold value may be 0.01 G, which is the smallest value of minute body movement registered in a body movement meter.

The arousal determining unit 34 determines that the subject is in an arousal state, if the body movement frequency determined by the body movement determining unit 33 is greater than a predetermined threshold value. The arousal determining unit 34 determines that the subject is in a sleep state, if the body movement frequency is smaller than the predetermined threshold value.

More specifically, the arousal determining unit 34 obtains the information as to whether there is body movement from the body movement determining unit 33, and measures the body movement frequency in a predetermined period. Here, the predetermined period is preferably one minute, for example. If the body movement frequency is greater than the predetermined threshold value, the subject is determined to be in an arousal state. If the body movement frequency is less than the predetermined threshold value, the subject is determined to be in a sleep state. The predetermined threshold value is preferably 20 times/minute, which is based on the movement frequency at the time of arousal in the past.

The sleep determining unit 35 determines whether the subject is in a sleep state, based on the autonomic nerve indexes LF and HF calculated by the autonomic nerve index calculating unit 32, and the determination result of the arousal determining unit 34. To determine whether the subject is in a sleep state, the depth of sleep is measured. Here, the depth of sleep is the index of how active the brain of the subject being tested is. In this embodiment, the depth of sleep is measured to determined whether the subject is in non-REM sleep or REM sleep. If the subject is determined to be in non-REM sleep, whether it is “light sleep” or “deep sleep” is determined.

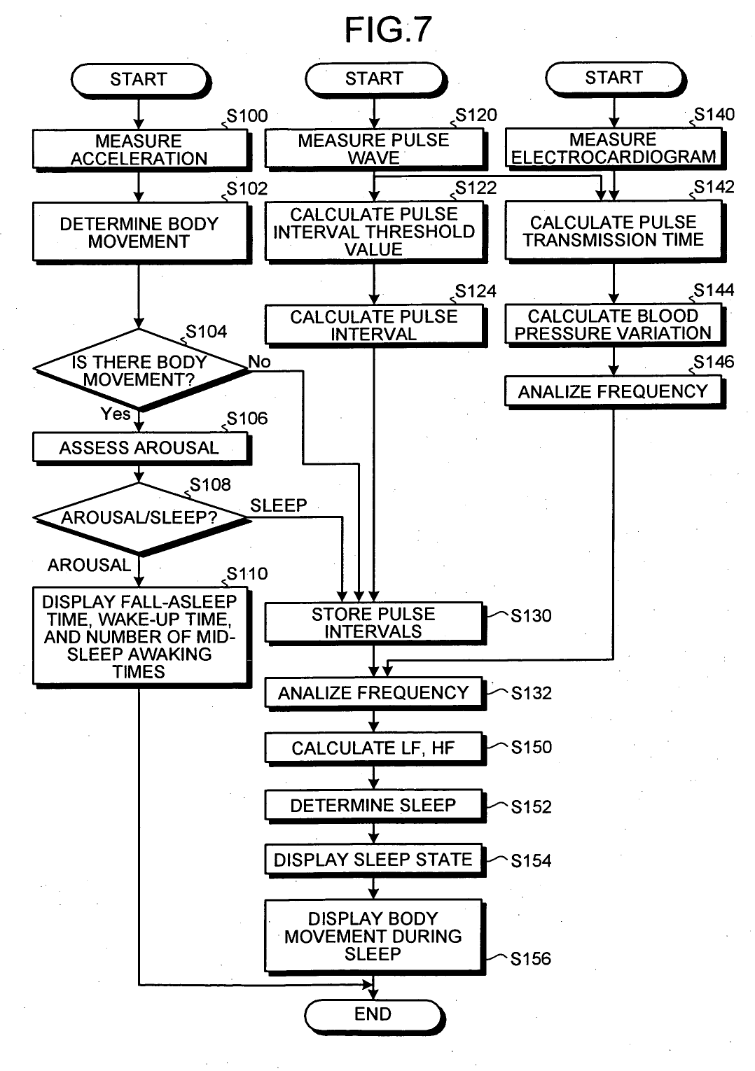

FIG. 7 is a flowchart of the health state managing operation of the health management apparatus 10. The health management apparatus 10 is attached to the subject to be tested prior to sleep, and the power supply and the health management functions are activated through the input unit 11. The acceleration measuring unit 21 starts the measurement of acceleration (step S100). The pulse wave measuring unit 18 starts measuring the pulse wave (step S120). The electrocardiogram measuring unit 15 starts electrocardiogram measurement (step S140).

As the acceleration measuring unit 21 starts measuring the acceleration, the body movement measuring unit 33 obtains body movement data from the acceleration data of the three axis directions obtained from the acceleration measuring unit 21. If the variation of the body movement data is greater than the threshold value, the body movement determining unit 33 detects body movement (step S102).

If the body movement determining unit 33 determines that there is body movement (Yes in step S104), the arousal determining unit 34 determines whether the subject being tested is in an arousal state or a sleep state (step S106).

If the body movement determining unit 33 determines that the subject being tested is in an arousal state (“Arousal” in step S108), the arousal determining unit 34 stores the fall-asleep time, the wake-up time, and the number of arousal during sleep in the memory unit 13. Further, the display unit 12 displays the fall-asleep time, the wake-up time, and the number of arousal during sleep (step S110).

Meanwhile, when the pulse wave measuring unit 18 starts measuring the pulse wave, the pulse interval calculating unit 31 calculates the pulse interval threshold value that is the dynamic threshold value for calculating the pulse intervals (step S122). The pulse interval calculating unit 31 next calculates the times when a series of pulse wave data corresponding to the threshold value appears from the series of pulse wave data from which the DC variation component has been removed, and sets the intervals between the calculated times as the pulse intervals (step S124).

Based on the result of the body movement determination in step S102 and the result of arousal determination in step S106, the pulse interval calculating unit 31 stores the pulse interval data only when the subject is in a sleep state and there is no body movement (step S130).

The pulse interval calculating unit 31 then converts the series of pulse interval data into a frequency spectrum distribution through frequency analysis such as an FFT method (step S132).

Meanwhile, when the electrocardiogram measuring unit 15 starts electrocardiogram measurement (step S140), the pulse transmission time calculating unit 30 calculates the pulse transmission time, based on the electrocardiogram measurement value and the pulse wave measurement value measured in step S120 (step S142). The pulse transmission time calculating unit 30 next converts the pulse transmission time into a blood pressure value (step S144). The blood pressure variability data is then converted into a frequency spectrum distribution by a frequency analysis method (step S146).

The autonomic nerve index calculating unit 32 calculates indexes LF and HF from the power spectrum values of the series of pulse interval data converted into the frequency spectrum distribution in step S132. The autonomic nerve index calculating unit 32 also calculates another index LF from the power spectrum values of the blood pressure variability data converted in step S146. The index HF calculated from the pulse interval data is then set as the autonomic nerve index. Based on the two indexes LF, the autonomic nerve index calculating unit 32 then determines an index LF to be an autonomic nerve index (step S150).

Next, the sleep determining unit 35 determines a sleep state, based on the autonomic nerve indexes LF and HF, and stores the determination result in the memory unit 13 (step S152). The display unit 12 then displays the sleep state (step S154), and also displays the amount of body movement during the sleep (step S156).

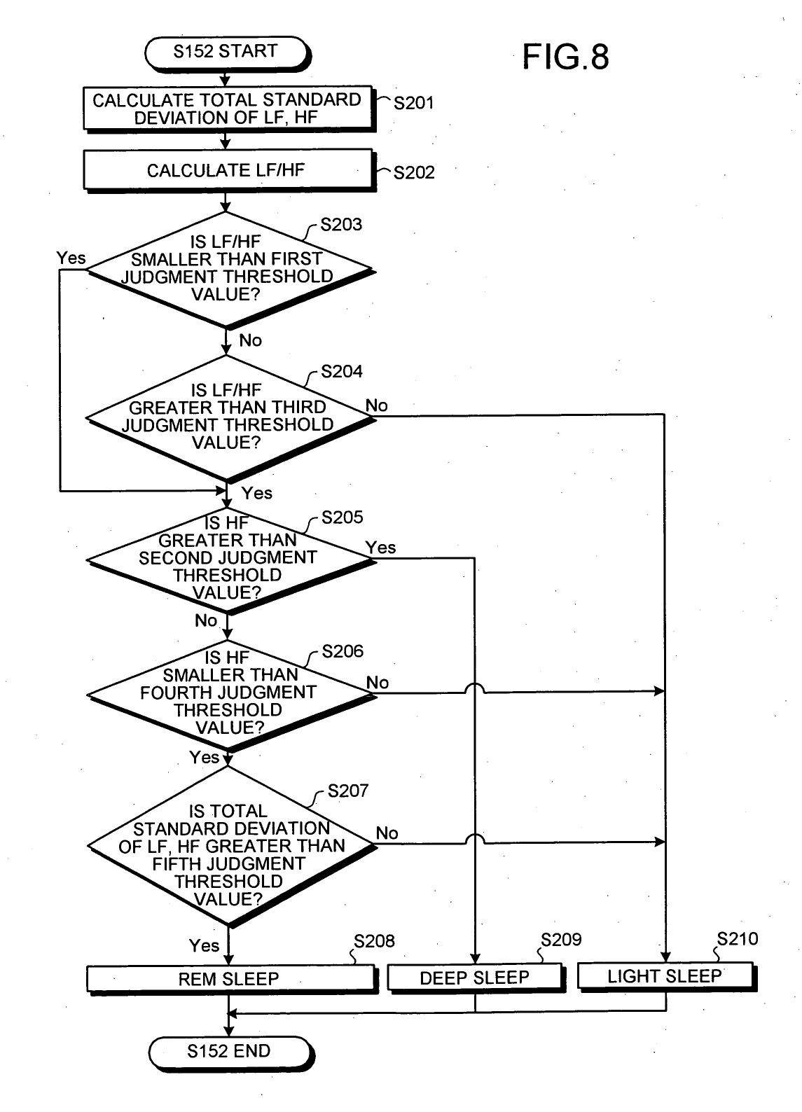

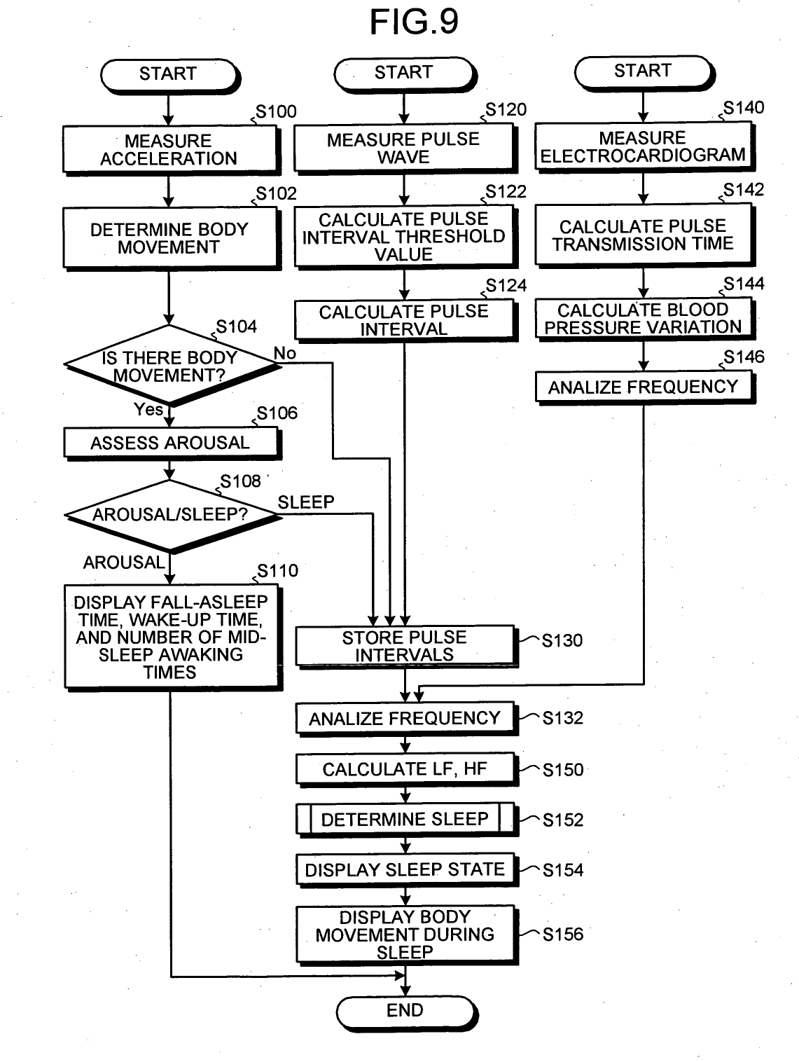

FIG. 8 is a flowchart of the procedures in step S152. In the following, the sleep determining operation of step S152 is explained in detail.

The sleep determining unit 35 first obtains the indexes LF and HF from the autonomic nerve index calculating unit 32, and calculates the total standard deviation of the indexes LF and HF (step S201). The sleep determining unit 35 also calculates the value of LF/HF (step S202).

Next, the sleep determining unit 35 determines whether the value of LF/HF is smaller than a first judgment threshold value (step S203). If the value of LF/HF is smaller than the first judgment threshold value (Yes in step S203), the sleep determining unit 35 further determines whether the value of HF is greater than a second judgment threshold value (step S205). If the value of HF is greater than the second judgment threshold value (Yes in step S205), the sleep determining unit 35 determines that the sleep is “deep sleep” (step S209).

If the value of LF/HF is equal to or greater than the first judgment threshold value (No in step S203), the sleep determining unit 35 further determines whether the value of LF/HF is greater than a third judgment threshold value (step S204). If the value of LF/HF is greater than the third judgment threshold value (Yes in step S204), the sleep determining unit 35 further determines whether the value of HF is greater than the second judgment threshold value (step S205)

If the value of HF is equal to or smaller than the second judgment threshold value (No in step S205), the sleep determining unit 35 further determines whether the value of HF is smaller than a fourth judgment threshold value (step S206). If the value of HF is smaller than the fourth judgment threshold value (Yes in step S206), the sleep determining unit 35 further determines whether the total standard deviation of the indexes LF and HF is greater than a fifth judgment threshold value (step S207). If the total standard deviation of the indexes LF and HF is greater than the fifth judgment threshold value (Yes in step S207), the sleep determining unit 35 determines that the sleep is “REM sleep” (step S208).

Meanwhile, if the value of LF/HF is equal to or smaller than the second judgment threshold value (No in step S204), and the value of HF is equal to or greater than the fourth judgment threshold value (No in step S206), and the total standard deviation of LF and HF is equal to or smaller than the fifth judgment threshold value (No in step S207), the sleep determining unit 35 determines that the sleep is “light sleep” (step S210).

The first to fifth judgment threshold values can be set by selecting two points with high distribution density of each of LF, HF, and LF/HF measured overnight for each subject, with the midpoint of the two points of LF/HF being the first judgment threshold value=the third judgment threshold value, the midpoint of the two points of HF being the second judgment threshold value=the fourth judgment threshold value, and the midpoint of the two points of LF being the fifth judgment threshold value.

As the acceleration data of the three axis directions are measured as body movement data, body movement data can be readily measured with high accuracy. Accordingly, the adverse influence of body movement on the pulse wave and the adverse influence of erratic pulse waves such as arrhythmia and anaerosis can be reduced, and the accuracy in determining a sleep state can be increased.

The health management apparatus 10 of the first embodiment has a hardware structure (not shown) including a read-only memory (ROM) that stores a health management program or the like for performing a health managing operation in the health management apparatus 10, and a CPU that controls each component of the health management apparatus 10 according to the program stored in the ROM.

The health management program in the health management apparatus 10 may be presented in a file of an installable format or an executable format that is recorded on a computer-readable recording medium such as a CD-ROM, a floppy disk (FD), or a DVD.

In such a case, a sleeping-time health management program is read out from the recording medium and executed, so that the program is loaded into the main storage device in the health management apparatus 10, and each of the components of the above described software structure is formed in the main storage device.

Alternatively, the sleeping-time health management program may be stored in a computer that is connected to a network such as the Internet, so that it can be downloaded via the network.

Although the electrocardiogram action potential is detected by measuring electrocardiogram movement in the first embodiment, a magneto cardiograph showing the electric activity of the heart may be taken by measuring the magnetism in the body, or the heart sound caused by the heartbeat may be measured.

Next, a health management apparatus according to a second embodiment of the present invention is explained. FIG. 9 is a flowchart of the sleep-time health managing operation of the health management apparatus according to the second embodiment. After the frequency analysis of step S146, the health management apparatus according to the second embodiment calculates an index LF (hereinafter referred to as the blood pressure index LF) based on the blood pressure variability value (step S150). In step S152, the sleep state is determined based on the indexes LF and HF calculated from the pulse intervals (hereinafter referred to as the pulse interval indexes LF and HF), and the blood pressure index LF.

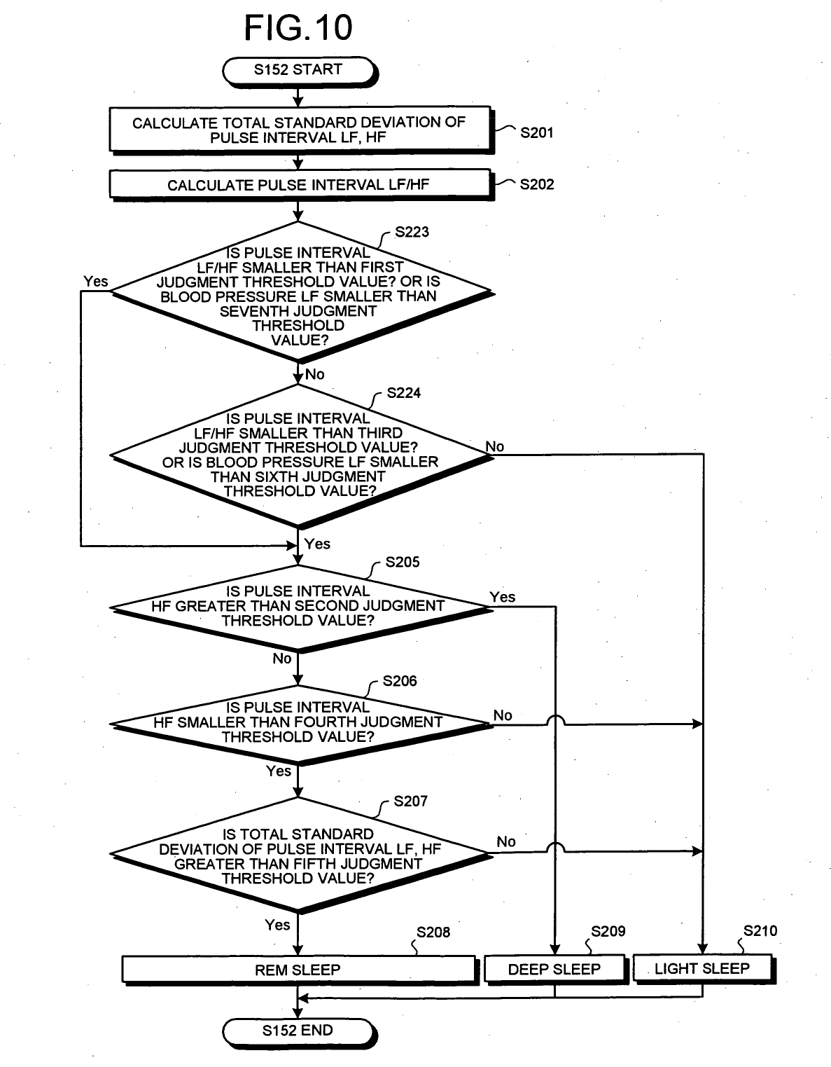

FIG. 10 is a flowchart of the operation in step S152 in the second embodiment.

In the second embodiment, if the value of the pulse interval index LF/HF is smaller than a first judgment threshold value or the blood pressure index LF is smaller than a seventh judgment threshold value (Yes in step S223), and the value of the pulse interval index HF is greater than a second judgment threshold value (Yes in step S205), the sleep is determined to be deep sleep (step S209).

If the value of the pulse interval index LF/HF is greater than a third judgment threshold value or the blood pressure index LF is greater than a sixth judgment threshold value (Yes in step S224), and the pulse interval index HF is smaller than the second judgment threshold value (Yes in step S205), and the total standard deviation of the pulse interval indexes LF and HF is greater than a fifth judgment threshold value (Yes in step S207), the sleep is determined to be REM sleep. Other than the cases of deep sleep and REM sleep, the sleep is determined to be light sleep (step S210).

The first to seventh judgment threshold values can be set by selecting two points with high distribution density of each of LF, HF, LF/HF, and the blood pressure index LF measured overnight for each subject, with the midpoint of the two points of LF/HF being the first judgment threshold value=the third judgment threshold value, the midpoint of the two points of HF being the second judgment threshold value=the fourth judgment threshold value, and the midpoint of the two points of LF being the fifth judgment threshold value.

The blood pressure value variation is one of the indexes of autonomic nerve activities. Accordingly, if the blood pressure value variation is wide, the sympathetic nerve is dominant. With the blood pressure value variation being used as a parameter for detecting REM sleep, the judgment accuracy can be increased.

The other aspects of the structure and operation of the health management apparatus according to the second embodiment are the same as those of the structure and operation of the health management apparatus 10 according to the first embodiment.

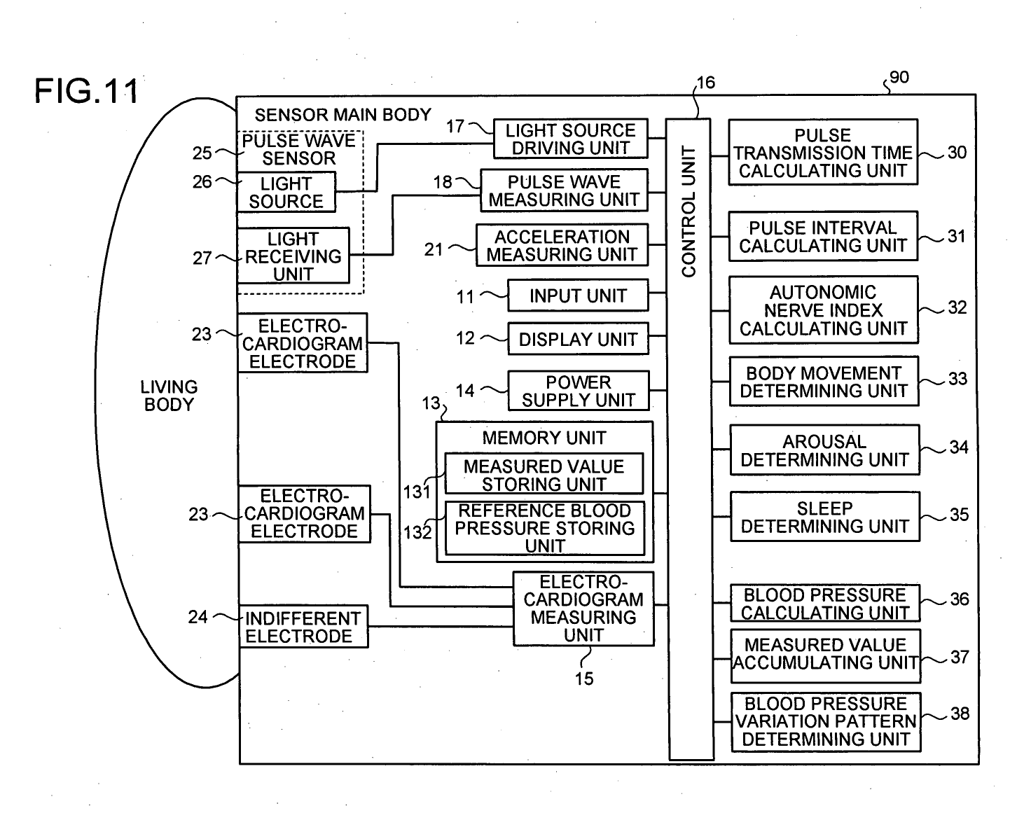

Next, a health management apparatus 90 according to a third embodiment of the present invention is explained. FIG. 11 is a block diagram showing the entire structure of the health management apparatus 90 according to the third embodiment. The health management apparatus 90 according to the third embodiment includes a blood pressure calculating unit 36, a measured value accumulating unit 37, and a pattern determining unit 38, as well as the same components as those of the health management apparatus 10 of the first embodiment. The memory unit 13 of the health management apparatus 90 according to the third embodiment includes a measured value storing unit 131 and a reference blood pressure storing unit 132.

The blood pressure calculating unit 36 calculates blood pressure, based on the pulse transmission time calculated by the pulse transmission time calculating unit 30. More specifically, a blood pressure value is calculated using equation (1) described in the first embodiment. The mean value of blood pressure values measured in a predetermined period of time is set as the blood pressure value. Here, the predetermined period of time may be 10 seconds, for example. When the constant α is determined, the reference blood pressure stored in the reference blood pressure storing unit 132 is used.

The blood pressure calculating unit 36 calculates a blood pressure value, based on the value measured at the same time as the measured value that is used by the sleep determining unit 35 determining a sleep state. Accordingly, the correspondence between the blood pressure value and the sleep state can be grasped.

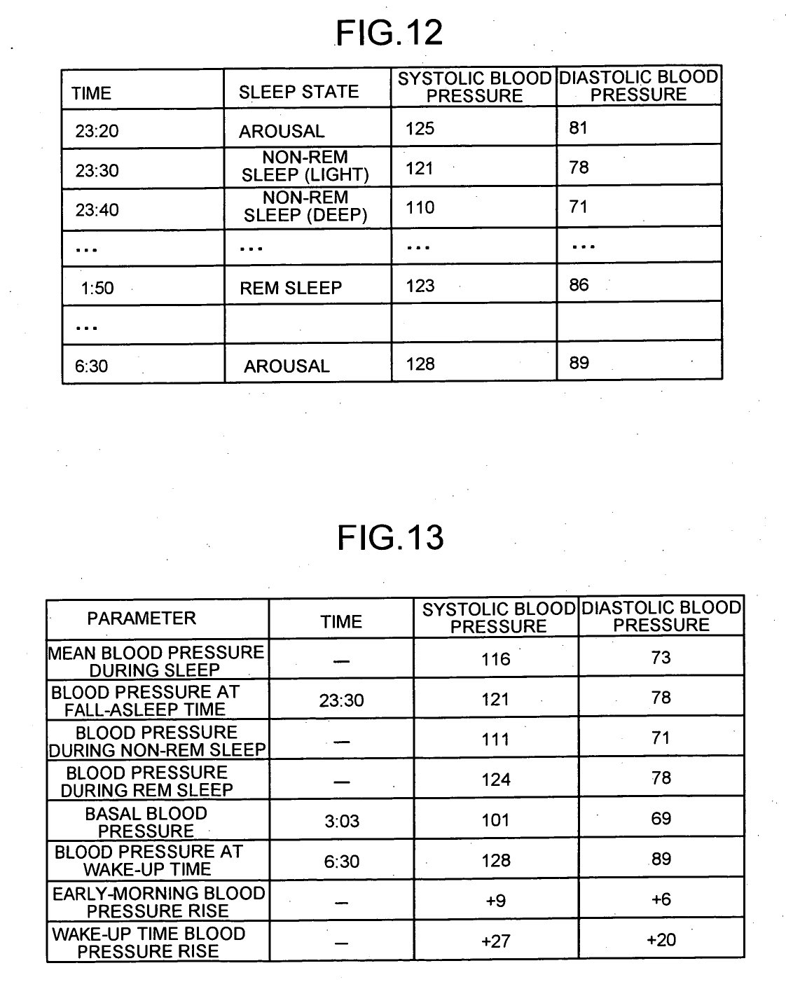

The measured value storing unit 131 stores each sleep state and each blood pressure value in association with each observed time and date. FIG. 12 schematically shows the data structure in the measured value storing unit 131. As shown in FIG. 12, the measured value storing unit 131 stores the sleep state, the systolic blood pressure, and the diastolic blood pressure in association with each measurement time. Each sleep state is a result of the determination of the sleep determining unit 35. Each systolic blood pressure and each diastolic blood pressure are values calculated by the blood pressure calculating unit 36.

The reference blood pressure storing unit 132 stores the reference blood pressure value of the subject being tested. The reference blood pressure value is obtained via the input unit 11 or a communication unit (not shown). In this embodiment, the blood pressure and the pulse transmission time prior to sleep are simultaneously measured with a conventional blood-pressure meter with cuffs (not shown). The measured blood pressure value is stored in the reference blood pressure storing unit 132.

Alternatively, the mean blood pressure value of the subject before sleep may be registered in the reference blood pressure storing unit 132 in advance. The reference blood pressure of the same subject does not greatly fluctuate. Accordingly, the mean value of the blood pressure can be regarded as the reference blood pressure. Further, the reference blood pressure stored in the reference blood pressure storing unit 132 may be corrected using the correlation with the amplitude of the pulse wave.

The measured value accumulating unit 37 accumulates the measured values that are stored in the measured value storing unit 131. For example, the measured value accumulating unit 37 adds up the measures values at the end of the measurement of the pulse wave and the likes, i.e., at the wake-up time of the subject. Alternatively, an accumulating operation may be performed every time a measured result is obtained.

FIG. 13 schematically shows the result of accumulation by the measured value accumulating unit 37. As shown in FIG. 13, the measured value accumulating unit 37 calculates the mean value of the systolic blood pressure during sleep and the mean value of the diastolic blood pressure during sleep. Also, the systolic blood pressure and the diastolic blood pressure are extracted when the subject being tested falls asleep and wakes up. The systolic blood pressure and the diastolic blood pressure are extracted during non-REM sleep and during REM sleep. Also, the time when basal blood pressure is measured, as well as the systolic blood pressure and the diastolic blood pressure at this time, are extracted.

Here, the basal blood pressure is the minimum blood pressure during non-REM sleep. After the subject falls asleep, the blood pressure normally lowers as the sympathetic nerves calm down. Especially during non-REM sleep, the blood pressure drops greatly. On the other hand, during REM sleep, the autonomic nerves are greatly disturbed. As a result, the blood pressure value fluctuates, and the mean blood pressure value during REM sleep increases. Also, under the control of circadian rhythm, the minimun blood pressure during non-REM sleep is the lowest blood pressure in one day. This is called the basal blood pressure.

A case with a high basal blood pressure and blood pressure that does not become lower even at night is called the non-dipper type. The ones of this type often have disorders in organs such as brains, hearts, and kidneys. Accordingly, the health of each subject being tested can be managed by showing the results of basal blood pressure measurement to the subject. A sharp decrease in basal blood pressure might cause hypoxic encephalopathy, and might be related to senile dementia. Therefore, basal blood pressure measurement is critically important.

In this embodiment, the minimum value among the blood pressure values during non-REM sleep determined by the sleep determining unit 35 is regarded as the basal blood pressure value. Alternatively, the minimum value among the blood pressure values obtained during the period from fall-asleep time till wake-up time may be set as the basal blood pressure. With this arrangement, even if the blood pressure values contain errors, an appropriate basal blood pressure value can be picked.

Also, the systolic blood pressure and the diastolic blood pressure are extracted early in the morning and at the wake-up time. Further, the blood pressure increase rate in the early morning and the blood pressure increase at the wake-up time are calculated.

The early-morning blood pressure rise is also known as early-morning high blood pressure, and increases the risk for circulatory system diseases. The early-morning blood pressure increase rate and the wake-up time blood pressure increase rate can serve as the indexes of early-morning high blood pressure.

The above calculation is now described in greater detail. First, the fall-asleep time blood pressure and the wake-up time blood pressure are extracted. When a sleep state lasts over three times (30 minutes) after arousal continues and sleep is detected for the first time, the time at which the sleep state is first detected is set as the fall-asleep time.

When arousal lasts over three times after a sleep state continues and arousal is detected for the first time, the time at which the arousal is first detected is set as the wake-up time. The blood pressure measured at the fall-asleep time is set as the fall-asleep time blood pressure. The blood pressure measured at the wake-up time is set as the wake-up time blood pressure.

The arousal determining unit 34 may be referred to as a wake-up determining unit.

Next, the mean blood pressure value is calculated with respect to each of sleep, REM sleep, and non-REM sleep. At the same time, the minimum blood pressure value during non-REM sleep is detected, and the time at which the minimum blood pressure value is detected is determined. The minimum blood pressure value is set as the basal blood pressure. Alternatively, the blood pressure measured when the minimum pulse wave value is obtained may be set as the basal blood pressure.

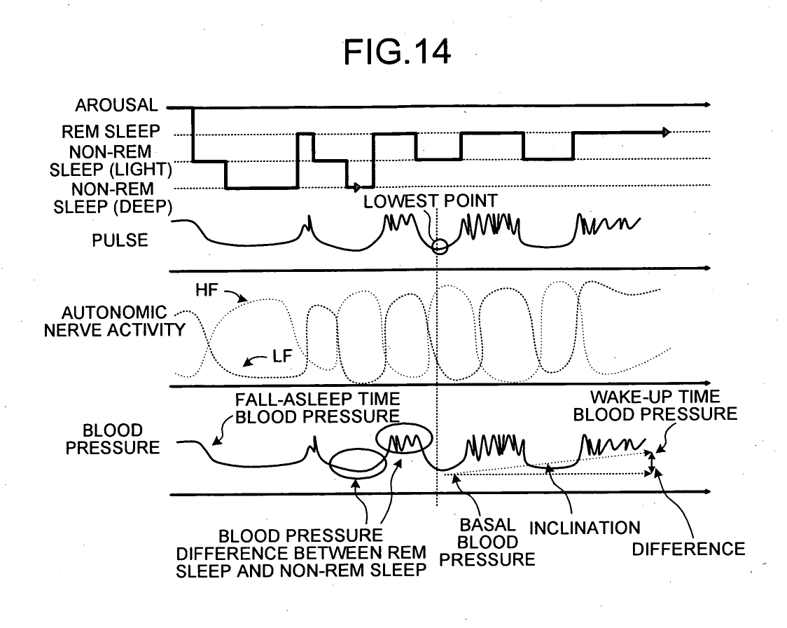

Here, the basal blood pressure is explained in greater detail. FIG. 14 shows mid-sleep blood pressure values associated with sleep states and autonomic nerve activities. As shown in FIG. 14, the blood pressure value varies in synchronization with the pulse. Accordingly, the basal blood pressure can be determined from either of the blood pressure values corresponding to the time of the minimum value of the blood pressure during non-REM sleep and the time of the minimum value of the pulse.

The measured value accumulating unit 37 further calculates the variation per unit of time, based on the basal blood pressure and the blood pressure during the last non-REM sleep, i.e., calculates the early-morning blood pressure increase rate. More specifically, the inclination of the regression line of blood pressure measured after the time when the basal blood pressure is measured may be set as the early-morning blood pressure increase rate. Also, the variation per unit of time calculated from the basal blood pressure and the blood pressure at the wake-up time, which is the wake-up time blood pressure increase rate is calculated. Likewise, the inclination of the regression line may be set as the wake-up time blood pressure increase rate.

Further, the blood pressure increase rate after the basal blood pressure is measured may be calculated. The blood pressure increase rate after the basal blood pressure is also valid as an index for early-morning high blood pressure.

The measured value accumulating unit 37 may be included in the health determining unit. The blood pressure variability pattern determining unit 38 may be configured to be divided into a pattern comparing unit and the health determining unit.

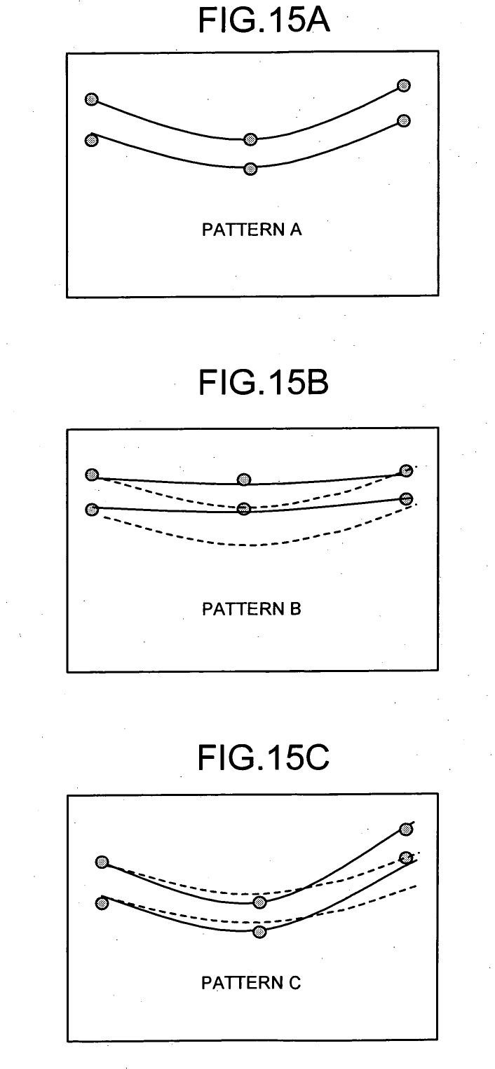

Referring back to FIG. 11, the pattern determining unit 38 determines a blood pressure variability pattern, based on the result of accumulation by the measured value accumulating unit 37. More specifically, blood variation patterns that are formed with the values of the fall-asleep time blood pressure, the basal blood pressure, and the wake-up time blood pressure in association with the measurement times of them is stored in advance. FIGS. 15A to 15C schematically show the blood variation patterns.

The pattern A shown in FIG. 15A is a pattern in which the basal blood pressure is the minimum value, and the fall-asleep time blood pressure is substantially equal to the wake-up time blood pressure. This blood pressure variability represents a healthy blood pressure variability.

The pattern B shown in FIG. 15B is a pattern in which the basal blood pressure does not become much lower than the fall-asleep time blood pressure and the wake-up time blood pressure. This blood pressure variability represents a possibility of disorders in the brain, the heart, or the liver.

The pattern C shown in FIG. 15C is a pattern in which the basal blood pressure becomes lower than the fall-asleep time blood pressure, but the wake-up time blood pressure is high. This blood pressure variability represents a high possibility of cardiac infarction or the like.

The pattern determining unit 38 compares the stored blood pressure variability patterns with the blood pressure value actually accumulated by the measured value accumulating unit 37, so as to select the blood pressure variability pattern closest to the actual blood pressure.

The pattern determining unit 38 also adds remarks for each of the blood pressure variability patterns, and stores them.

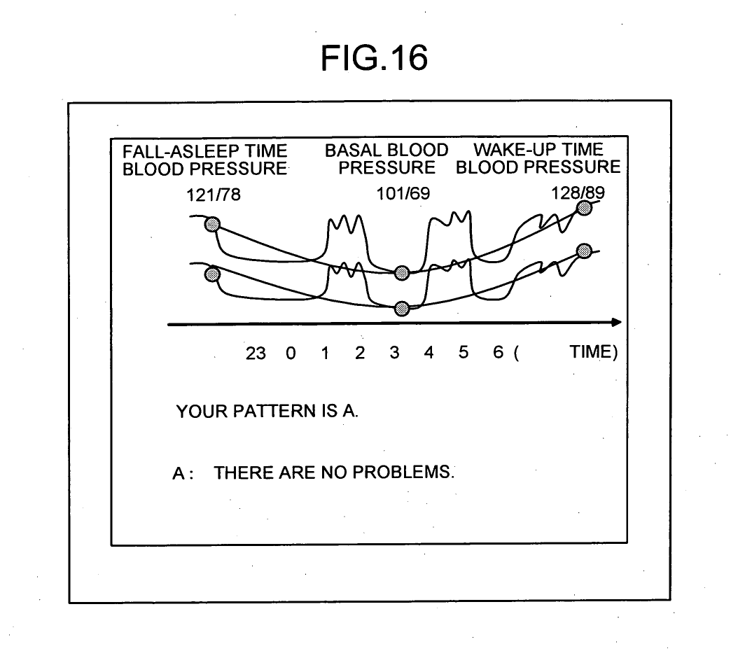

FIG. 16 shows an example of the screen displayed on the display unit 12 when the blood pressure variability pattern is the pattern A. As shown in FIG. 16, the screen shows the graph of the measurement results, the selected blood pressure variability pattern, and the remark for the selected blood pressure variability pattern. Here, the graph of the measurement results is formed by plotting the fall-asleep time blood pressure, the basal blood pressure, and the wake-up time blood pressure.

In the case of the pattern B, the display unit 12 may display the remark “The basal blood pressure has not dropped much; see a doctor to check a possibility of disorders in the brain, the heart, and the kidneys.” In the case of the pattern C, the display unit 12 may display the remark “The blood pressure increase rate is high in the morning; see a doctor as soon as possible, because there is a high possibility of cardiac infarction or the like.”

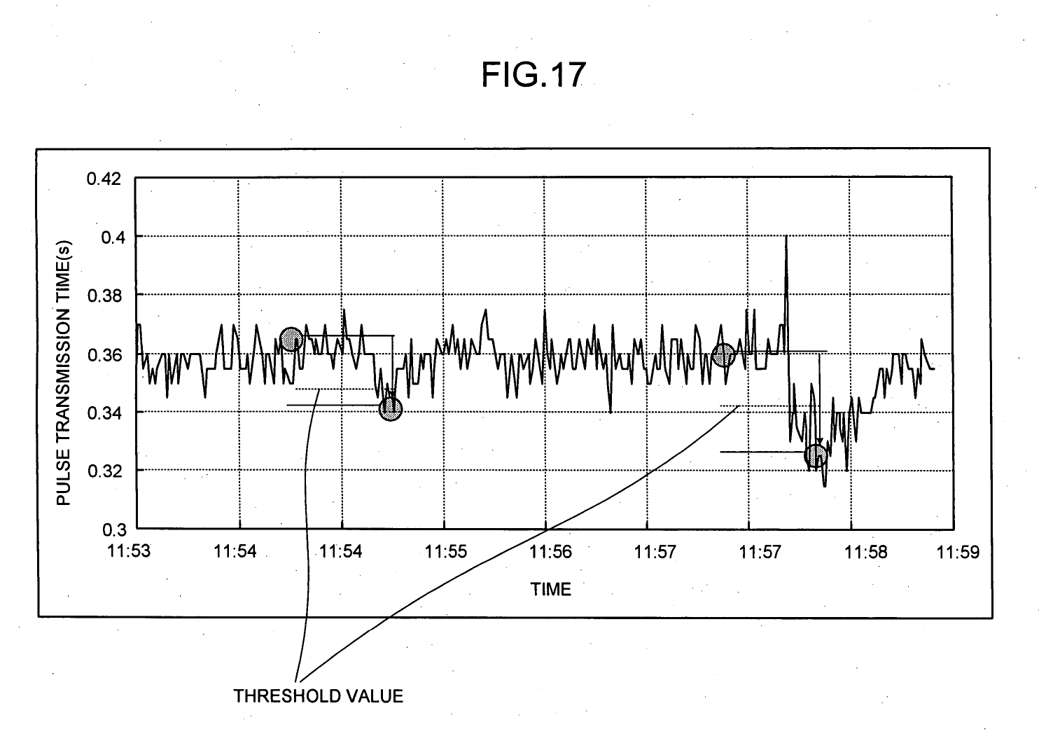

The measured value accumulating unit 37 further calculates the rate of change of the pulse transmission time in a predetermined period of time. FIG. 17 shows the operation of calculating the rate of change of the pulse transmission time. With the pulse transmission time at a predetermined clock time being set as the reference value, a predetermined value is set as the threshold value. More specifically, “pulse transmission time ±0.02 s” is set as the threshold value, and the number of times when the pulse transmission time exceeds the threshold value between the predetermined clock time and another predetermined clock time is counted.

In a case of a sleep apnea syndrome, the frequency of blood pressure variability tends to increase. Since the pulse transmission time is inversely proportional to the blood pressure, the index for a sleep apnea syndrome can be obtained from the counted number.

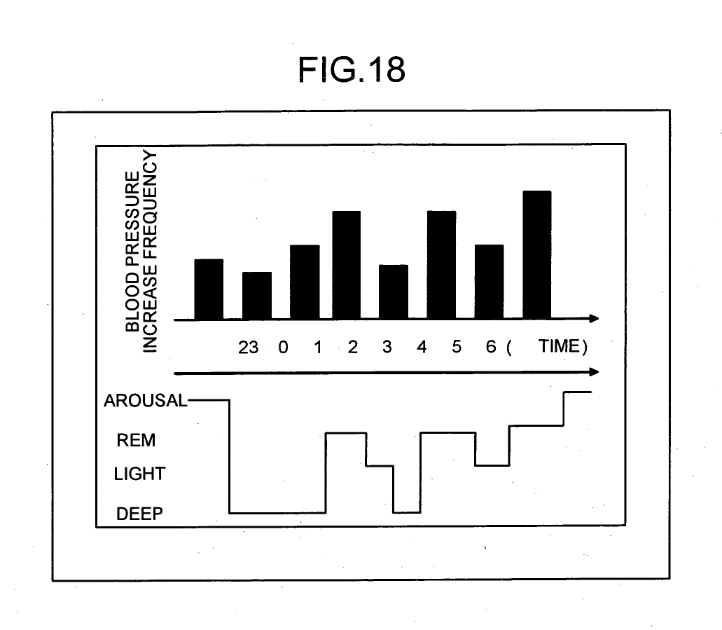

FIG. 18 is a graph of the counted number (the blood pressure increase frequency) by the hour. The measured value accumulating unit 37 causes the display unit 12 to display the graph of FIG. 18. By doing so, the subject being tested can obtain the index for a sleep apnea syndrome.

The other aspects of the structure and operation of the health management apparatus 90 of the third embodiment are the same as those of the structure and operation of the health management apparatus 10 of the first embodiment.

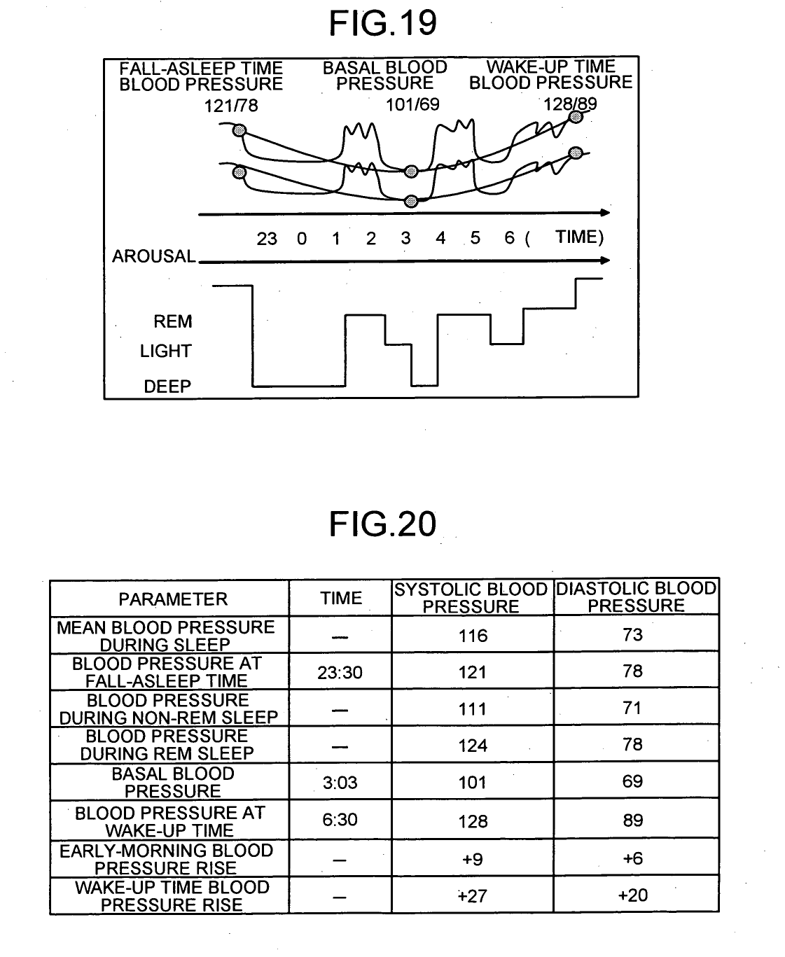

It is also possible for the display unit 12 to display the sleep state, as well as the blood pressure variability pattern, as shown in FIG. 19.

Alternatively, the display unit 12 may display the accumulated values stored in the reference blood pressure storing unit 132 in the form of a table, as shown in FIG. 20. In this case, the values outside the effective range may be indicated in bold print or the like. The effective range is stored in the memory unit 13.

Blood pressure values vary greatly with the positions and the heights of the measured parts. Therefore, the influence may be corrected. More specifically, the position of each measured part is detected by the acceleration sensor, and the blood pressure value is calculated based on the correction coefficient.

One correction coefficient is prepared beforehand for each position to be detected, and is stored in the memory unit 13. A position determining unit (not shown) then determines each position, based on the output of the acceleration sensor. Positions to be determined include sitting positions, standing positions, spine positions, and lateral positions. The correction coefficient associated with the detected position is retrieved from the memory unit 13, and the blood pressure value is corrected by multiplying the blood pressure value determined from the pulse transmission time by the correction coefficient.

After the basal blood pressure is calculated, the measured value accumulating unit 37 monitors the early-morning blood pressure increase rate. If the blood pressure increase rate is high, the subject being tested may be notified of the fact. More specifically, the measured value accumulating unit 37 calculates the blood pressure increase rate per unit of time, every time a unit of time has passed. If the blood pressure increase rate is higher than a predetermined value, an alarm is output to the subject being tested.

Further, threshold values are set for the blood pressure increase rate. When the blood pressure increase rate exceeds the lowest threshold value, the subject being tested is notified of the result. When the blood pressure increase rate exceeds a higher threshold value, the family of the subject being tested is notified of the result. When the blood pressure increase rate exceeds an even higher threshold value, the management company is notified of the result. More specifically, the health management apparatus 90 has a communication function to inform the family of the fact that the blood pressure increase rate exceeds the threshold value through a mobile communication device. Also, it is possible to inform the management company of the fact through a communication terminal. However, the communication methods are not limited to this.

As described above, with the health management apparatus 90 of the third embodiment, the blood pressure during non-REM sleep, during which the autonomic nerves particularly calm down, can be measured. Accordingly, accurate blood pressure (the basal blood pressure) can be measured, without adverse influence of external disturbance that cannot be avoided with a conventional blood-pressure meter.

Also, the body positions during sleep are measured to calculate a more accurate early-morning blood pressure increase rate, a more accurate wake-up time blood pressure increase rate, and a blood pressure varying frequency in a predetermined period of time. Thus, the sign of a disorder such as early-morning high blood pressure, high blood pressure during REM sleep, and high blood pressure during apnea can be detected.

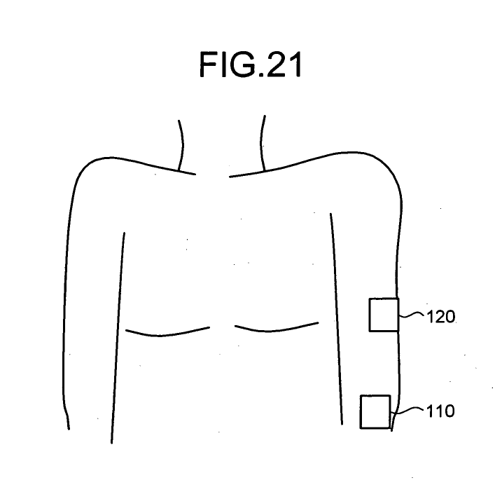

Next, a health management system 100 according to a fourth embodiment of the present invention is explained. FIG. 21 shows an example of how the health management system 100 of the fourth embodiment is attached to a subject to be tested. The health management system 100 of the fourth embodiment includes a health management main apparatus 110 and a health management sub apparatus 120. The health management main apparatus 110 and the health management sub apparatus 120 measure the pulse waves at two different parts of the subject.

In the example shown in FIG. 21, the health management main apparatus 110 is attached to a wrist of the subject. The health management sub apparatus 120 is attached to an elbow. Based on the difference in distance, the pulse transmission time is determined. The health management system 100 of the fourth embodiment that measures the pulse waves at two different part of the subject being tested differs from each health management apparatus of the first to third embodiments that measures the electrocardiogram data and the pulse wave.

To calculate the pulse transmission time, two different parts with two different propagation times should be measured, and the measured parts and the measurement methods are not limited to the examples in this embodiment.

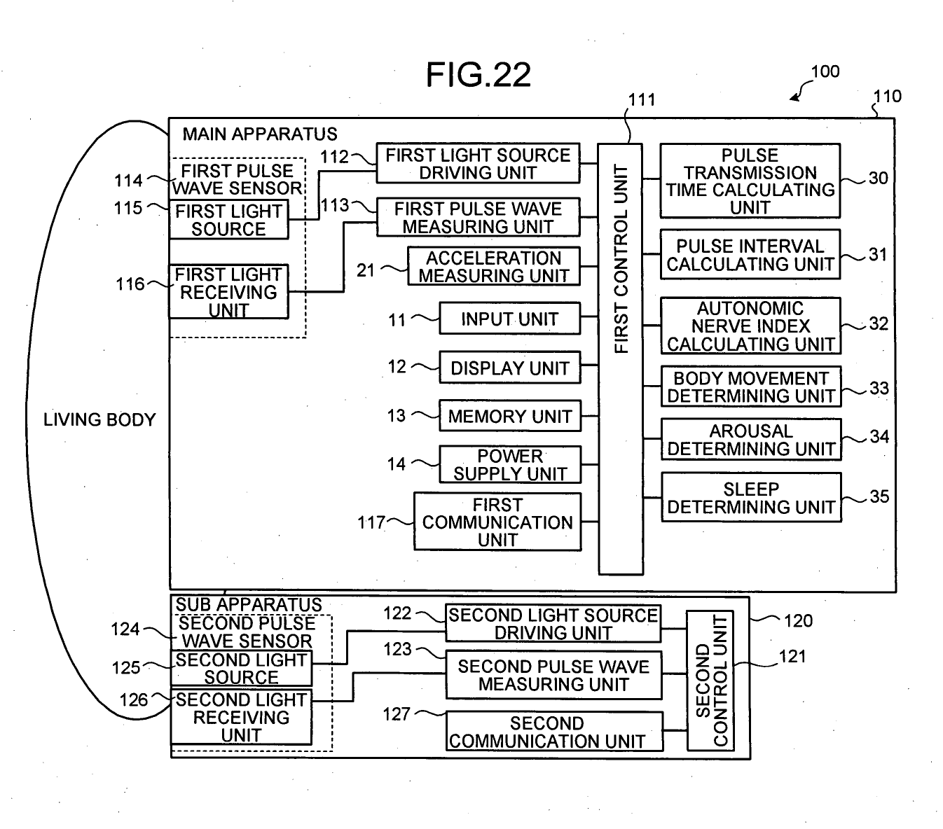

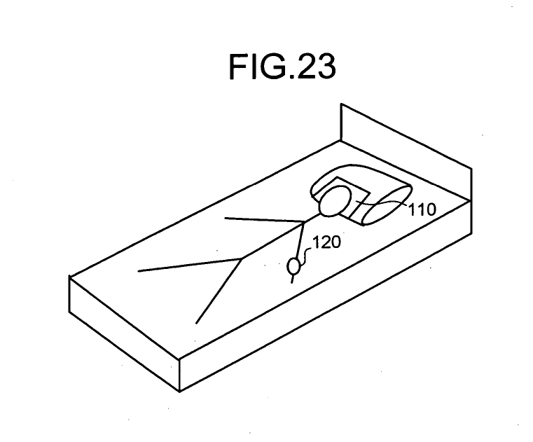

FIG. 22 is a block diagram showing the entire structure of the health management system 100 according to the fourth embodiment. The health management main apparatus 110 of the health management system 100 of the fourth embodiment includes an input unit 11, a display unit 12, a memory unit 13, a power supply unit 14, an electrocardiogram measuring unit 15, a first control unit 111, a first light source driving unit 112, a first pulse wave measuring unit 113, an acceleration measuring unit 21, a first pulse wave sensor 114 including a first light source 115 and a first light receiving unit 116, a first communication unit 117, a pulse transmission time calculating unit 30, a pulse interval calculating unit 31, an autonomic nerve index calculating unit 32, a body movement determining unit 33, an arousal determining unit 34, and a sleep determining unit 35.

The health management sub apparatus 120 of the health management system 100 includes a second control unit 121, a second light source driving unit 122, a second pulse wave measuring unit 123, a second pulse wave sensor 124 including a second light source 125 and a second light receiving unit 126, and a second communication unit 127.

The first light source driving unit 112, the first pulse wave measuring unit 113, the first light source 115, and the first light receiving unit 116 perform the same operations as the light source driving unit 17, the pulse wave measuring unit 18, the light source 26, and the light receiving unit 27 of the first embodiment.

The first communication unit 117 and the second communication unit 127 exchange information. The first control unit 111 of the health management main apparatus 110 transmits a sampling control signal for matching the sampling timings to the health management sub apparatus 120 via the first communication unit 117.

The second light source driving unit 122, the second pulse wave measuring unit 123, the second light source 125, and the second light receiving unit 126 perform the same operations as the light source driving unit 17, the pulse wave measuring unit 18, the light source 26, and the light receiving unit 27 of the first embodiment.

The second control unit 121 obtains the sampling control signal via the second communication unit 127. With the sampling control signal being a trigger, the second control unit 121 performs the driving of the second light source 125 and A-D conversion. By doing so, two pulse waves can be measured in the same timing. The second control unit 121 also processes signals obtained through the second light receiving unit 126. The processed signals are transmitted to the health management main apparatus 110 via the second communication unit 127.

The first control unit 111 obtains the pulse wave of the elbow from the health management sub apparatus 120 via the first communication unit 117. The pulse wave of the elbow associated with the pulse wave of the wrist measured by the first pulse wave measuring unit 113 at the same time is stored in the memory in the first control unit 111. The pulse transmission time calculating unit 30 obtains the signal of the pulse wave of the elbow and the signal of the pulse wave of the wrist stored in the first control unit 111. Peak detection from each signal is then performed. The time difference between the detection of the peak of the elbow pulse wave and the detection of the peak of the wrist pulse wave is determined as the pulse transmission time.

The other aspects of the structure and operation of the health management system 100 of the fourth embodiment are the same as those of the structure and operation of the health management apparatus 10 of the first embodiment. It is of course possible to apply the method of measuring the pulse waves at two parts to the second embodiment or the third embodiment.

Next, a health management system 190 according to a fifth embodiment of the present invention is explained. FIG. 23 shows an example of how the health management system 190 of the fifth embodiment is attached to a subject to be tested. The health management system 190 of the fifth embodiment includes a health management main apparatus 110 and a health management sub apparatus 120. The health management main apparatus 110 of the fifth embodiment measures pressure pulse waves that are detected by a pressure sensor.

In this embodiment, the health management main apparatus 110 is contained in a pillow, and detects the pressure pulse wave of the neck of the subject. The health management sub apparatus 120 detects the pulse wave of a wrist of the subject. The time difference between the pressure pulse wave of the neck and the pulse wave of the wrist is calculated as the pulse transmission time.

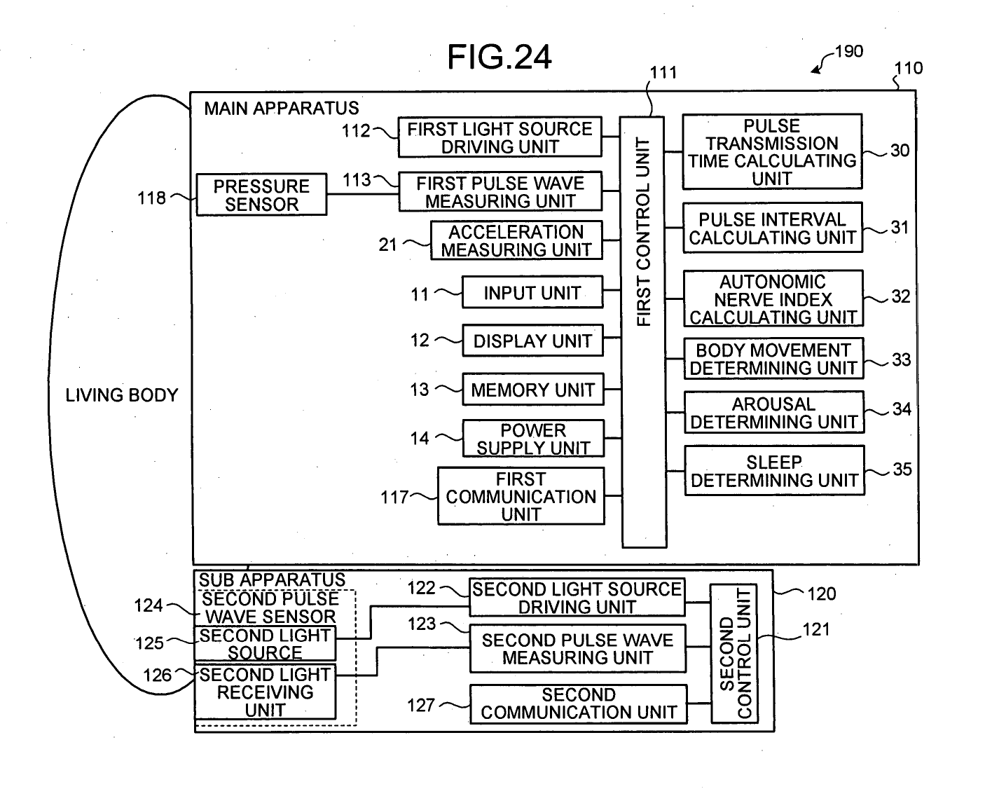

FIG. 24 is a block diagram showing the entire structure of the health management system 190 according to the fifth embodiment. The health management main apparatus 110 of the health management system 190 has a pressure sensor 118, instead of the first light source driving unit 112 and the first pulse wave sensor 114 of the health management main apparatus 110 of the fourth embodiment.

The first pulse wave measuring unit 113 is connected to the pressure sensor 118. Piezoelectric transformation is performed on the pressure variation of the neck detected by the pressure sensor 118, followed by analog signal processing such as filtering. The pressure pulse wave is then A-D converted, and is transferred to the first control unit 111. The first control unit 111 associates the pressure pulse wave with the pulse wave of the wrist detected at the same time, and the pressure pulse wave and the pulse wave are stored in the memory in the first control unit 111.

The other aspects of the structure and operation of the health management system 190 of the fifth embodiment are the same as those of the structure and operation of the health management system 100 of the fourth embodiment.

Instead of the second pulse wave sensor 124 of the health management sub apparatus to be attached to a wrist, an electrocardiogram electrode to be attached to the chest region of a subject may be employed. Also, it is possible to employ an electrocardiograph with a single electrode to be attached to a wrist. In the case of using electrocardiogram, the time difference between the electrocardiogram peak and the peak of the pressure pulse wave of the neck region is set as the pulse transmission time.

Although the health management main apparatus 110 of this embodiment is contained in a pillow and detects the pressure pulse wave of the neck region of the subject, it may be contained in a bed. In such a case, the pressure sensor 118 detects the pressure pulse wave of the chest region. The health management main apparatus 110 may also be placed under or on the mattress. In such a case, the pressure sensor 118 detects the pressure pulse wave of the chest region of the subject being tested. The operation of the health management main apparatus 110 in this case is the same as the operation of the health management main apparatus 110 of the fourth embodiment.

Although the embodiments of the present invention have been described so far, various changes and modifications may be made to those embodiments.

In a first modification, the health management apparatus 10 of the first embodiment may calculate the pulse transmission time based on the pulse wave measurement values at two different points, instead of an electrocardiogram measurement value and a pulse wave measurement value, like the health management system 100 of the fourth embodiment. The same applies to the second and third embodiments.

In a second modification, the health management system 100 of the fourth embodiment may calculate the pulse transmission time based on an electrocardiogram measurement value and a pulse wave measurement value, instead of the pulse wave measurement values at two different points, like the health management apparatus 10 of the first embodiment.

In this case, the health management main apparatus 110 may measure an electrocardiogram measurement value, while the health management sub apparatus 120 measures a pulse wave. Alternatively, the health management main apparatus 110 may measure a pulse wave, while the health management sub apparatus 120 measures an electrocardiogram measurement value. The same applies to the fifth embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A health management apparatus comprising:
a first pulse wave measuring unit that measures a first pulse wave of a subject during sleep;
a second pulse wave measuring unit that measures a second pulse wave of the subject during sleep, the second pulse wave being different from the first pulse wave in propagation time from the heart of the subject;
a pulse transmission time calculating unit that calculates a pulse transmission time indicating a time difference between the first pulse wave and the second pulse wave;
a pulse interval calculating unit that calculates a pulse interval based on at least one of the first pulse wave and the second pulse wave;
an autonomic nerve index calculating unit that calculates an autonomic nerve index indicating an autonomic nerve activity of the subject based on the pulse transmission time and the pulse interval; and
a health determining unit that determines the condition of health of the subject based on the autonomic nerve index.

2. The health management apparatus according to claim 1, further comprising a health output unit that outputs the condition of health.

3. The health management apparatus according to claim 1, wherein the health determining unit determines a sleep state based on the autonomic nerve index.

4. The health management apparatus according to claim 3, wherein the health determining unit determines whether the sleep is REM sleep or non-REM sleep based on the autonomic nerve index.

5. The health management apparatus according to claim 1, wherein the autonomic nerve index calculating unit calculates the autonomic nerve index in a low-frequency region based on the pulse transmission time.

6. The health management apparatus according to claim 1, further comprising a pressure sensor that detects pressure from the body weight of the subject,
wherein the first pulse wave measuring unit measures the first pulse wave based on the pressure detected by the pressure sensor.

7. A health management apparatus comprising:
a first pulse wave measuring unit that measures a first pulse wave of a subject during sleep;
a second pulse wave measuring unit that measures a second pulse wave of the subject during sleep, the second pulse wave being different from the first pulse wave in propagation time from the heart of the subject;
a pulse transmission time calculating unit that calculates a pulse transmission time indicating a time difference between the first pulse wave and the second pulse wave;
a pulse interval calculating unit that calculates a pulse interval based on at least one of the first pulse wave and the second pulse wave;
an autonomic nerve index calculating unit that calculates an autonomic nerve index indicating an autonomic nerve activity of the subject based on the pulse transmission time and the pulse interval;
a blood pressure value calculating unit that calculates a blood pressure value based on the pulse transmission time; and
a health determining unit that determines the condition of health of the subject based on the autonomic nerve index and the blood pressure value.

8. The health management apparatus according to claim 7, further comprising:
a blood pressure variability pattern storing unit that stores a blood pressure variability pattern indicating a time variation in blood pressure during sleep; and
a pattern comparing unit that compares the time variation in blood pressure during sleep with the blood pressure variability pattern,
wherein the health determining unit determines the condition of health of the subject based on the comparison result.

9. The health management apparatus according to claim 8, further comprising a sleep determining unit that determines whether the sleep is REM sleep or non-REM sleep based on the autonomic nerve index,
wherein the pattern comparing unit compares the time variation in blood pressure during sleep with the blood pressure variability pattern, the blood pressure during sleep being obtained when the sleep determining unit detects REM sleep.

10. The health management apparatus according to claim 7, further comprising a basal blood pressure determining unit that determines a basal blood pressure to be a minimum value in the time variation of the blood pressure value,
wherein the health determining unit determines the condition of health based on the basal blood pressure.

11. The health management apparatus according to claim 7, further comprising:
a sleep determining unit that determines whether the sleep is REM sleep or non-REM sleep based on the autonomic nerve index; and
a basal blood pressure determining unit that determines a basal blood pressure to be a minimum value of the blood pressure obtained when the sleep determining unit detects REM sleep,
wherein the health determining unit determines the condition of health based on the basal blood pressure.

12. The health management apparatus according to claim 10, further comprising:
a wake-up determining unit that determines whether the subject has woken up based on the autonomic nerve index; and
a wake-up time blood pressure increase rate calculating unit that calculates a blood pressure increase rate from the basal blood pressure to the blood pressure value obtained when the subject is determined to has waken up,
wherein the health determining unit determines the condition of health of the subject based on the blood pressure increase rate.

13. The health management apparatus according to claim 11, further comprising:
a wake-up determining unit that determines whether the subject has woken up based on the autonomic nerve index; and
a wake-up time blood pressure increase rate calculating unit that calculates a blood pressure increase rate from the basal blood pressure to the blood pressure value obtained when the subject is determined to has waken up,
wherein the health determining unit determines the condition of health of the subject based on the blood pressure increase rate.

14. The health management apparatus according to claim 10, further comprising:
a sleep determining unit that determines whether the sleep is REM sleep or non-REM sleep based on the autonomic nerve index; and
an early-morning blood pressure increase rate calculating unit that calculates a blood pressure increase rate from the basal blood pressure to the blood pressure value obtained when the last non-REM sleep during the sleep is detected,
wherein the health determining unit determines the condition of health of the subject based on the blood pressure increase rate.

15. The health management apparatus according to claim 7, further comprising a pulse transmission time variation measuring unit that measures a variation in a predetermined period of time within the pulse transmission time,
wherein the health determining unit determines the condition of health of the subject based on a result of the measurement by the pulse transmission time variation measuring unit.

16. The health management apparatus according to claim 7, further comprising a pressure sensor that detects pressure from the body weight of the subject,
wherein the first pulse wave measuring unit measures the first pulse wave based on the pressure detected by the pressure sensor.

17. A health management apparatus comprising:
an electrocardiogram activity measuring unit that measures the degree of an electrocardiogram activity of a subject during sleep;
a first pulse wave measuring unit that measures a first pulse wave of the subject during sleep;
a pulse transmission time calculating unit that calculates a pulse transmission time indicating a time difference between the electrocardiogram activity and the first pulse wave;
a pulse interval calculating unit that calculates a pulse interval based on at least one of the degree of the electrocardiogram activity and the first pulse wave;
an autonomic nerve index calculating unit that calculates an autonomic nerve index indicating an autonomic nerve activity of the subject based on the pulse transmission time and the pulse interval; and
a health determining unit that determines the condition of health of the subject based on the autonomic nerve index.

18. A health management apparatus comprising:
an electrocardiogram activity measuring unit that measures the degree of an electrocardiogram activity of a subject during sleep;
a first pulse wave measuring unit that measures a first pulse wave of the subject during sleep;
a pulse transmission time calculating unit that calculates a pulse transmission time indicating a time difference between the electrocardiogram activity and the first pulse wave;
a pulse interval calculating unit that calculates a pulse interval based on at least one of the degree of the electrocardiogram activity and the first pulse wave;
an autonomic nerve index calculating unit that calculates an autonomic nerve index indicating an autonomic nerve activity of the subject based on the pulse transmission time and the pulse interval;
a blood pressure value calculating unit that calculates a blood pressure value based on the pulse transmission time; and
a health determining unit that determines the condition of health of the subject based on the autonomic nerve index and the blood pressure value.

19. A health management system comprising a health management main apparatus and a health management sub apparatus that manage the condition of health of a subject,
the health management sub apparatus comprising:
a first pulse wave measuring unit that measures a first pulse wave of the subject during sleep; and
a transmission unit that transmits the first pulse wave to the health management main apparatus,
the health management main apparatus comprising:
a reception unit that receives the first pulse wave from the health management sub apparatus;
a second pulse wave measuring unit that measures a second pulse wave of the subject during sleep, the second pulse wave being different from the first pulse wave in propagation time from the heart of the subject;
a pulse transmission time calculating unit that calculates a pulse transmission time indicating a time difference between the first pulse wave and the second pulse wave;
a pulse interval calculating unit that calculates a pulse interval based on at least one of the first pulse wave and the second pulse wave;
an autonomic nerve index calculating unit that calculates an autonomic nerve index indicating an autonomic nerve activity of the subject based on the pulse transmission time and the pulse interval; and
a health determining unit that determines the condition of health of the subject based on the autonomic nerve index.

20. A health management system comprising a health management main apparatus and a health management sub apparatus that manage the condition of health of a subject,
the health management sub apparatus comprising:
a first pulse wave measuring unit that measures a first pulse wave of the subject during sleep; and
a transmission unit that transmits the first pulse wave to the health management main apparatus,
the health management main apparatus comprising:
a reception unit that receives the first pulse wave from the health management sub apparatus;
a second pulse wave measuring unit that measures a second pulse wave of the subject during sleep, the second pulse wave being different from the first pulse wave in propagation time from the heart of the subject;
a pulse transmission time calculating unit that calculates a pulse transmission time that is the time difference between the first pulse wave and the second pulse wave;
a pulse interval calculating unit that calculates a pulse interval based on at least one of the first pulse wave and the second pulse wave;
an autonomic nerve index calculating unit that calculates an autonomic nerve index indicating an autonomic nerve activity of the subject based on the pulse transmission time and the pulse interval;
a blood pressure value calculating unit that calculates a blood pressure value, based on the pulse transmission time; and
a health determining unit that determines the condition of health of the subject based on the autonomic nerve index and the blood pressure value.

21. A health management method comprising:
measuring a first pulse wave of a subject during sleep;
measuring a second pulse wave of the subject during sleep, the second pulse wave being different from the first pulse wave in propagation time from the heart of the subject;
calculating a pulse transmission time indicating a time difference between the first pulse wave and the second pulse wave;
calculating a pulse interval, based on at least one of the first pulse wave and the second pulse wave;
calculating an autonomic nerve index indicating an autonomic nerve activity of the subject based on the pulse transmission time and the pulse interval; and
determining the condition of health of the subject, based on the autonomic nerve index.

22. A health management method comprising:
measuring a first pulse wave of a subject during sleep;
measuring a second pulse wave of the subject during sleep, the second pulse wave being different from the first pulse wave in propagation time from the heart of the subject;
calculating a pulse transmission time indicating a time difference between the first pulse wave and the second pulse wave;
calculating a pulse interval based on at least one of the first pulse wave and the second pulse wave;
calculating an autonomic nerve index indicating an autonomic nerve activity of the subject based on the pulse transmission time and the pulse interval;
calculating a blood pressure value, based on the pulse transmission time; and
determining the condition of health of the subject, based on the autonomic nerve index and the blood pressure value.

23. A computer program product having a computer readable medium including programmed instructions for managing health, wherein the instructions, when executed by a computer, cause the computer to perform:
measuring a first pulse wave of a subject during sleep;
measuring a second pulse wave of the subject during sleep, the second pulse wave being different from the first pulse wave in propagation time from the heart of the subject;
calculating a pulse transmission time indicating a time difference between the first pulse wave and the second pulse wave;
calculating a pulse interval, based on at least one of the first pulse wave and the second pulse wave;
calculating an autonomic nerve index indicating an autonomic nerve activity of the subject based on the pulse transmission time and the pulse interval; and
determining the condition of health of the subject, based on the autonomic nerve index.

24. A computer program product having a computer readable medium including programmed instructions for managing health, wherein the instructions, when executed by a computer, cause the computer to perform:
measuring a first pulse wave of a subject during sleep;
measuring a second pulse wave of the subject during sleep, the second pulse wave being different from the first pulse wave in propagation time from the heart of the subject;
calculating a pulse transmission time indicating a time difference between the first pulse wave and the second pulse wave;
calculating a pulse interval based on at least one of the first pulse wave and the second pulse wave;
calculating an autonomic nerve index indicating an autonomic nerve activity of the subject based on the pulse transmission time and the pulse interval;
calculating a blood pressure value, based on the pulse transmission time; and
determining the condition of health of the subject, based on the autonomic nerve index and the blood pressure value.
